(19) United States
(12) Patent Application Publication
(10) Pub. No.: US 2022/0193256 A1
(43) Pub. Date: Jun. 23, 2022

(54) NANO-ANTIBIOTICS BASED ON SINGLE-CHAIN DEXTRAN NANOPARTICLES

(71) Applicant: FUNDACIÓN CIDETEC, Donostia-San Sebastián (ES)

(72) Inventors: Raquel GRACIA ESPAÑA, Donostia-San Sebastián (ES); Julie MOVELLAN, Donostia-San Sebastián (ES); Marco MARRADI, Donostia-San Sebastián (ES); Damien DUPIN, Donostia-San Sebastián (ES); lraida LOINAZ BORDONABE, Donostia-San Sebastián (ES); Xabier MURGIA ESTEVE, Donostia-San Sebastián (ES)

(21) Appl. No.: 17/604,590

(22) PCT Filed: Apr. 17, 2020
(86) PCT No.: PCT/EP2020/060804

§ 371 Date: Oct. 18, 2021

(30) Foreign Application Priority Data
Apr. 18, 2019 (EP) 19382309.3

(51) Int. Cl. A61K 47/69; A61K 47/61

(52) U.S. Cl. CPC A61K 47/6939; A61K 47/61

(57) ABSTRACT

The present invention relates to a water-dispersible single-chain dextran methacrylate or acrylate based nanoconjugate, which comprises a water-dispersible single-chain dextran methacrylate or acrylate based nanoparticle and one or more hydrophilic active ingredients, and having: a particle size from 3 nm to 50 nm, a zeta potential from −25 mV to +20 mV, a percentage of intra-molecular crosslinking from 3% to 45% molar % of the total amount of monomer units present in the dextran methacrylate or acrylate chain, and a drug loading from 20% to 40% by weight of the drug in relation to the total weight of the nanoconjugate. It also relates to processes for their preparation, compositions containing them and their use in therapy.

This application claims the benefit of European Patent Application 19382309.3 filed Apr. 18, 2019

The present invention relates to the field of single-chain dextran methacrylate or acrylate based nanoparticles and more particularly to the field of single-chain dextran methacrylate or acrylate based nanoconjugates. The present invention also relates to processes for their preparation and compositions containing them, as well as their use in therapy.

### BACKGROUND ART

Antibiotics are used to treat bacterial infections but it is well known that the use of antibiotics has been steadily increasing. However, the development of bacterial resistance is one of the most critical public health threats. In addition to this, the lack of promising new antibiotic classes under advanced clinical development have prompted the search for alternative strategies to improve the antimicrobial efficacy, prevent bacterial resistance mechanisms and extend the targeting or prolonged action of already existing drugs.

It is known in the state of the art that the most common used antibiotics for the treatment of bacterial infections, particularly lower respiratory track infections, are hydrophilic compounds with positive charges in their structure. These positive charges interact with the mucus (that is negatively charged) decreasing or preventing the appropriate diffusion of the antibiotic in the target site. In this way, improvement of the antibiotics delivery is a good strategy to fight this problem because it can reduce side effects and increase the efficacy protecting antibiotics from deactivation.

It has been disclosed the use of polymeric carriers to encapsulate antibiotics. Thus, antibiotic encapsulation into polymeric nanoparticle as carriers (also named nanoconjugates) has emerged as a promising strategy for the treatment of bacterial infections, particularly for the preparation of inhaled compositions containing antibiotic nanoconjugates for the treatment of lower respiratory track infections. However, unlike oncologic or gene therapies, antibiotics have to be administered in a fixed posology and in high quantities to patients, and therefore, high loading capabilities in the carriers are required.

Unfortunately, the conjugation (encapsulation) efficiency of antibiotics (hydrophilic active ingredients) in polymeric hydrophobic nanoparticles is generally low, resulting in an unappropriate low antibiotic loadings (<3% w/w) and as a result, large amounts of polymeric nanoparticles (nanocarriers) containing the antibiotic are needed to achieve the therapeutically effective amount of the antibiotic after being administered.

Without being bound to any theory, it seems that this low conjugation efficiency is mainly due to the mismatch between the hydrophilic nature of many antibiotics and the hydrophobic nature of polymers forming the nanoparticles (carriers).

Single-stranded nanoparticles (also named single-chain nanoparticles or SCNPs) have been described in the state of the art as suitable carriers for conjugating active ingredients. However, their loading capability is also one of their limiting aspect.

In order to solve the loading capability of SCNPs, different strategies have been used. Some of these strategies are focused in the use of hydrogen bonds. In particular, hydrogen bonds to load anticancer drugs in the core of hydrophobic polymeric particles stabilized by hydrophobic polyethylene (PEG) polymer chains has been disclosed. Similar strategy was employed for the encapsulation of rifampicin in the core of hydrophobic polymeric particles. However, the use of this strategy does not allow increasing the loading of the active ingredients enough for avoiding the administration of high amounts of the carrier for achieving the therapeutic effective amount of the active ingredient.

Other strategies used for increasing the percentage of loading involved the use of hydrophilic SCNPs having a hydrophilic nature such as for example SCNP based on methacrylic acids (cf. EP3215545). These SCNPs contains a high number of negative charges allowing a priori a higher loading of hydrophilic active ingredients. Unfortunately, the SCNPs based on methacrylic acids allows high loading but hinder an appropriate release from the nanoparticles. Therefore, the active ingredient is permanently linked to the SCNPs based on methacrylic acids; and being not appropriate for as a carriers of hydrophilic compounds such as antibiotics. Furthermore, hydrophilic SCNPs having lower content of negative charges were used for conjugating contract agents and vaccine. Unexpectedly, the loading of these active agents were still very low, particularly less than 10% by weight of loading.

Furthermore, other strategy disclosed in the state of the art is the use of hydrophilic SCNPs having hydrophilic nature such as for example SCNP based on dextran. These SCNPs contains a hydrolysable amide bond at the conjugation site between the active ingredient and the dextran single-chain nanoparticle. These SCNPs allows loading both hydrophobic active ingredient (i.e. doxorubicin; Tauvod. “Doxorubicin-Functionalized Dextran-Based Single Chain Polymeric Nanoparticles as New Potential . . . .” 2016, pages 1-8, URL:https://www.youtube.com/watch?v=g4_iHiOSvBo) and hydrophilic active ingredients by the used of an additional medium-length spacer between the hydrolysable amide bond and the active ingredient (i.e. NODA; Gracia et al. Journal of Materials Chemistry B, 2017, vol. 5, no. 6, pages 1143-1147). Unfortunately, the loading of these active agents were still very low, particularly less than 10% by weight of loading. Following this strategy, these SCNPs containing a hydrolysable amide bond at the conjugation site between the active ingredient and the dextran single-chain nanoparticle and an additional large-length spacer between the active ingredient and the dextran single-chain nanoparticle has been used for increasing the loading up to 25%. Unfortunately, the use of the additional spacer increase the particle size promoting the aggregation between nanoparticles and as a result compromising their therapeutic use.

Related to this fact, it is important to mention that another handicap of the use of polymeric nanoparticles for encapsulating active ingredients and particularly antibiotics is the control of the release of the active ingredient from the carrier. Common antibiotic treatment involves a strict posology of the drug, particularly with two or three intakes per day of high amounts of antibiotic per intake. Therefore, antibiotics treatment which allows a reduction in the number of intakes are advantageous for the adherence of the treatment. However, low antibiotic loading as mentioned above in turn contributes to have very slow antibiotic release profiles because there is an insufficient driving force for drug diffusion. Meanwhile, a high antibiotic loading does not necessary involve an appropriate release. It is known that there are multiple factors that can modify or hinder the release of the drug from the vehicle, such as the nature of the carrier, the active ingredient and the type of link between them.

Therefore, from what is known in the art, it is derived that there is still the need of providing a high loading polymeric nanoconjugate for hydrophilic active ingredients, particularly antibiotics, having an appropriate release from the conjugate and suitable distribution in the target site for being use in therapy.

### SUMMARY OF THE INVENTION

Inventors have found a single-chain dextran methacrylate or acrylate based nanoconjugate that contains a water-dispersible single-chain dextran methacrylate or acrylate based nanoparticle which allows a high loading of one or more hydrophilic active ingredients. Besides, inventors have found that the nanoconjugates of the invention allow an appropriate release of the hydrophilic active ingredient from the SCPNs for being used in therapy.

Without being bound to any theory, it seems that the combination of the degree of functionalization of the nanoparticle and the amount of intra-molecular crosslinking of the nanoconjugates of the present invention allows having high drug loading without compromising their release. The nanoconjugates of the present invention are particularly advantageous because they allow reducing the amount of nanocarrier administered for having the therapeutically effective amount of the hydrophilic active ingredient.

Furthermore, the nanoconjugates of the present invention wherein the drug and the nanoparticle are linked by non-covalent interactions (bonds) that allows having a faster release profile of the hydrophilic active ingredient from the nanoconjugates of the present invention than those having a hydrolysable covalent bond linking the drug and the nanoparticle. It means that the release of the hydrophilic ingredient from the nanoconjugates of the present invention can be modulated, i.e. it can be modified/adapted to the specific circumstances of the disease and/or patient.

Moreover, the nanoconjugates of the present invention, which have a small particle size and suitable zeta potential, have an appropriate diffusion without interacting with the mucus and arriving to the target site (lower respiratory track, particularly lungs).

Finally, as it is shown in the examples, the nanoconjugates of the present invention are appropriate for conjugating a high variability of hydrophilic active ingredients, and particularly hydrophilic antibiotics.

Thus, the first aspect of the present invention relates to a water-dispersible single-chain dextran methacrylate or acrylate based nanoconjugate, which comprises:
a water-dispersible single-chain dextran methacrylate or acrylate based nanoparticle and
one or more hydrophilic active ingredients,

and the nanoconjugate comprises units of formula (I)

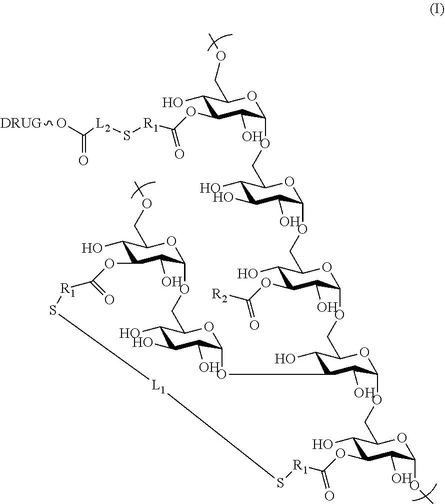

wherein: R₁ is selected from the group consisting of —CH₂—CH₂— and —CH₂—CH(CH₃)—; R₂ is selected from the group consisting of —CH(═CH₂), —C(═CH₂)(CH₃), —CH₂—CH₂—S—(CH₂)ᵥ—COOH, —CH(CH₃)—CH₂—S—(CH₂)ᵥ—COOH, —CH₂—CH₂—S—[(CH₂)_{w}—O]ₓ—(CH₂)_{w}—OH, and —CH(CH₃)—CH₂—S—[(CH₂)_{w}—O]ₓ—(CH₂)_{w}—OH; L₁ is a biradical selected from the group consisting of —[(CH₂)ᵣ—O]_{q}—(CH₂)ᵣ— and —(CH₂)ₛ—; L₂ is a biradical selected from the group consisting of —(CH₂)ᵥ— and —(C₅-C₆)arylene-NH—CO—(CH₂)ₚ—; DRUG is the hydrophilic active ingredient; p and v are integers from 1 to 6; w is an integer from 2 to 4; x is an integer from 0 to 4; q is an integer from 2 to 3; r is an integer from 2 to 4; and s is an integer from 2 to 5; [symbol] represents a non-covalent bond between the negative charge of the oxygen atom of the group —O—CO— of the nanoparticle and the positive charge of a nitrogen atom of the drug; or alternatively a hydrolysable covalent bond between the oxygen atom of the group —O—CO— of the nanoparticle and a carbon atom of the drug; wherein the nanoconjugate has: a particle size from 3 nm to 50 nm measured by Dynamic Light Scattering; a zeta potential from −25 mV to +20 mV; a percentage of intra-molecular crosslinking from 3% to 45% molar % of the total amount of monomer units present in the dextran methacrylate or acrylate chain; and a drug loading from 20% to 40% by weight of the drug in relation to the total weight of the nanoconjugate.

The second aspect of the present invention relates to a process for the preparation of the water-dispersible single-chain dextran methacrylate or acrylate based nanoconjugate of the first aspect of the invention.

The third aspect of the invention relates to a pharmaceutical composition which comprises the water-dispersible dextran methacrylate or acrylate based nanoconjugate as defined in the first aspect of the invention, together with one or more pharmaceutically acceptable excipients or carriers.

The fourth aspect of the invention relates to the water-dispersible dextran methacrylate or acrylate based nanoconjugate as defined in the first aspect of the invention, for use in therapy.

Finally, the fifth aspect of the invention relates to the use of the water-dispersible dextran methacrylate or acrylate based nanoconjugate as defined in the first aspect of the invention for use in the treatment of bacterial infection, when the hydrophilic active ingredient is a hydrophilic antibiotic.

### BRIEF DESCRIPTION OF DRAWINGS

FIG. 1 shows the pharmacokinetic profile of free tobramycin (white squares) and Tob-DXT-SCPN1-ionic of Example 9 of the present invention (black circles). In particular, it represents the mean tobramycin levels per lung pair homogenate (expressed as ng of Tobramycin/lung pair) versus time (expressed in hours).

FIG. 2 shows the mean percentage of tobramycin that remains in the lungs homogenate of animals either treated with free tobramycin (white squares) or Tob-DXT-SCPN1-ionic of Example 9 of the present invention (black circles) as a function of the nominal dose. In particular, it represents the individual amount of tobramycin administered to each animal (expressed as percentage (%) of the nominal dose) in lungs versus time (expressed in hours).

### DETAILED DESCRIPTION OF THE INVENTION

All terms as used herein in this application, unless otherwise stated, shall be understood in their ordinary meaning as known in the art. Other more specific definitions terms as used in the present application are as set forth below and are intended to apply uniformly throughout the specification and claims unless an otherwise expressly set out definition provides a broader definition.

For the purposes of the present invention, any ranges given include both the lower and the upper end-points of the range. Ranges given, such as temperatures, times, weights, and the like, should be considered approximate, unless specifically stated.

The terms “percentage (%) by weight”, “weight/weight %” and “w/w %” have the same meaning and are used interchangeably. They refer to the percentage of one ingredient in relation to the total weight of a composition or mixture.

The term “molar %” as used herein refers to the mole fraction or molar fraction, which is the amount of an ingredient expressed in moles, divided by the total amount of monomer units present in the dextran methacrylate or acrylate chain.

The term “water-dispersible” refers to a single-chain dextran methacrylate or acrylate based nanoparticle or nanoconjugate whose dispersability in water is equal to or greater than 100 mg per litre of water. The term “dispersability” refers to the capacity of the single-chain dextran methacrylate or acrylate based nanoparticle or nanoconjugate of the present invention to be uniformly dispersed in water and can be filtered through a 0.2 micron size filter.

The term “aqueous solution” refers to a solution which contains minimum 50 wt % of pure water.

The term “dextran” or “dextran chain” have the same meaning and are used interchangeably. They refer to a complex branched glucan (polysaccharide derived from the condensation of glucose). IUPAC defines dextrans as “branched poly-α-d-glucosides of microbial origin having glycosidic bonds predominantly C-1→C-6”. Dextran chains are of varying molecular weights (from 3 to 2000 kilodaltons). The polymer main chain of dextran consists of α-1,6 glycosidic linkages between glucose monomers, with branches from α-1,3 linkages.

The term “dextran methacrylate or acrylate based” refers to a nanoparticle or nanoconjugate formed by a dextran that include esters of methacrylate or acrylate in the structure.

In particular, the methacrylate esters are usually prepared by transesterification by treating the dextran with glycidyl methacrylate (GMA) under such reaction conditions to form an ester bond from a hydroxyl group of the glucose monomer and the carbonyl group of the GMA (cf. van Dijk-Wolthuis et al., Biomolecular Engineering, 2007, vol. 24, pp. 496-504) as it is summarized in the scheme below:

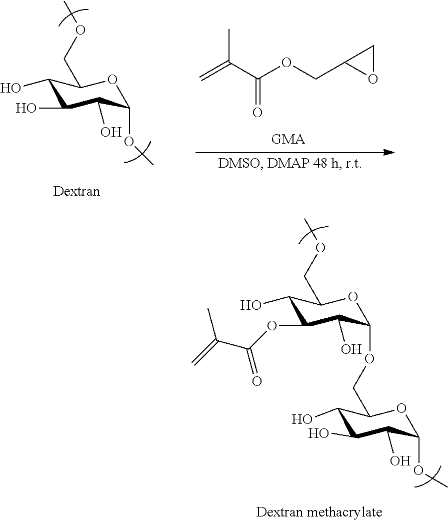

The acrylate esters are usually prepared from a slightly modified procedure which implies treating the dextran with vinyl acrylate (VA) under such reaction conditions to form an ester bond from a hydroxyl group of the glucose monomer and the carbonyl group of the VA (cf. L. Ferreira et al., Biomaterials, 2002, vol. 23, pp. 3957-3967) as it is summarized in the scheme below:

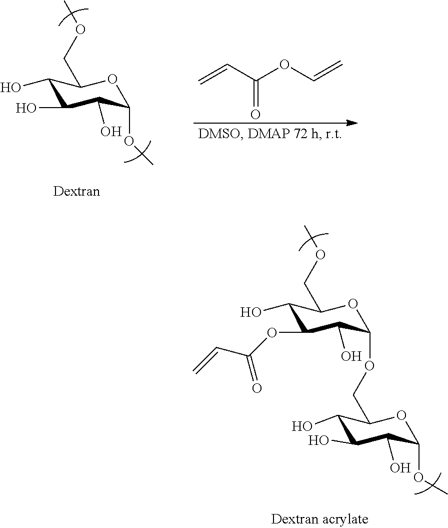

The dextran methacrylate or acrylate polymer used as starting material in the present invention have a solubility equal to or higher than 100 mg per litre of water. The solubility of the dextran methacrylate or acrylate can be measured by any method disclosed in the state of the art for measuring the solubility of polymers. In particular, in the present invention the solubility has been determined measuring visible light transmittance which has to be 100% for those completely dissolved dextran methacrylate or acrylate.

The dextran methacrylate or acrylate used in the present invention as a starting material contain an amount of methacrylate or acrylate groups from 5 to 60 molar % of the total amount of monomer units present in the dextran chain. The “methacrylate or acrylate groups” allow forming a three-dimensional crosslinked nanoparticle (network) by reaction with the crosslinkable groups of the crosslinking agent. The “methacrylate or acrylate groups” can be forming part of the backbone of the dextran polymer, or can be attached to the backbone as lateral chains. The methacrylate or acrylate groups can be forming part of the structure of the dextran or they can be introduced in the structure of the dextran. When the methacrylate or acrylate groups are introduced, this step can be carried out in several steps, either during the polymerization step, or when the polymerization process has already finished. The amount of methacrylate or acrylate groups present in the dextran chain can be measured by Nuclear Magnetic Resonance (NMR) spectroscopy. Particularly by proton nuclear magnetic resonance spectroscopy.

The term “polydispersion of a polymer (dextran methacrylate or acrylate polymer) or “polydispersion of a nanoparticle” (dextran methacrylate or acrylate based SCPN) refers to the value calculated by dividing the weight average molecular weight of the polymer by the number average molecular weight. This value means the range of molecular weights present in the dextran methacrylate or acrylate polymer and refers to the broadness of the molecular weight distribution obtained from size exclusion chromatography (SEC) analysis.

The terms “polydispersity of a nanoparticle” refers to the polydispersity index of the particle size (PDI (size)), a value obtained from the square of the standard deviation divided by the mean diameter. The polydispersity of the nanoparticles size refers to the broadness of the size distribution obtained by Dynamic Light Scattering (DLS).

The term “crosslinked” refers to a nanoparticle or nanoconjugate that have three-dimensional crosslink network wherein the network is formed by a single-chain of the starting dextran methacrylate or acrylate polymer that has been collapsed by one or more crosslinking agents.

The term “crosslinking” refers in the polymer science field to the use of cross-links to promote a difference in the physical properties of the polymers. The term “crosslink” refers to bonds that link one “complementary reactive group” methacrylate or acrylate groups of the dextran polymer) with a crosslinkable group of the crosslinking agent by hydrolysable covalent bonding. For example, the bond that link one “complementary reactive group” methacrylate or acrylate reactive groups) of the single-chain of the polymer with a crosslinkable group of the crosslinking agent by hydrolysable covalent bonding. The term “crosslinker” or “crosslinking agent” which is herein used interchangeably refers to compound having the ability to cross-link polymer chain(s). The term “homobifunctional crosslinking agent” refers to those crosslinking agents that contain two identical reactive sites (i.e. two identical crosslinkable groups), which can react with the “complementary reactive groups” methacrylate or acrylate groups of dextran).

As it is mentioned above, the water-dispersible single-chain dextran methacrylate or acrylate based nanoconjugate of the first aspect of the invention comprises a water-dispersible single-chain dextran methacrylate or acrylate based nanoparticle and one or more hydrophilic active ingredients, and the nanoconjugate comprises units of formula (I) as defined in the present invention.

The nanoconjugate of the present invention has a particle size from 3 nm to 50 nm measured by Dynamic Light Scattering. In an embodiment, the water-dispersible single-chain dextran methacrylate or acrylate based nanoconjugate has a particle size from 10 nm to 50 nm; particularly from 15 nm to 35 nm. These nanoconjugates are especially advantageous because they can be administered by inhalation. The particle size of the nanoparticles used as starting material and the nanoconjugates of the invention can be measured by any method disclosed in the state of the art for measuring the particle size of nanoparticles. Commonly, the particle size and the polydispersity index of the nanoparticle (PDI size) is measured by Dynamic light scattering (DLS) reporting the Z-average diameter value.

The term “zeta potential” refers to the electrokinetic potential in colloidal systems, which means the charge that develops at the interface between a particle surface and its dispersant. In an embodiment, the water-dispersible single-chain dextran methacrylate or acrylate based nanoconjugate has a zeta potential from −25 mV to +20 mV. In an embodiment, the water-dispersible single-chain dextran methacrylate or acrylate based nanoconjugate is one wherein [symbol] represents a non-covalent bond as defined in the present invention and the positive charge of a nitrogen atom of the drug and the zeta potential from −25 mV to +5 mV; particularly from −15 mV to +5 mV. In an embodiment, the water-dispersible single-chain dextran methacrylate or acrylate based nanoconjugate is one wherein [symbol] represents a hydrolysable covalent bond as defined in the present invention and the zeta potential from −15 mV to +20 mV; particularly from −10 mV to +20 mV and more particularly from −5 mV to +20 mV. The zeta potential can be measured by any appropriate method known in the state of the art. In particular, the method used in the present invention is mixed mode measurement, phase analysis light scattering (M3-PALS) using a Zetasizer Nano ZS apparatus.

The term “non-covalent” bond refers to the bond between the nanoparticles and the hydrophilic drug that involves weak interactions such as for example electrostatic interactions, hydrogen bonding and/or van der Waals interactions. In an embodiment, the non-covalent bond is an ionic interaction, electrostatic interaction between a negative charge of the nanoparticle and a positive charge of the hydrophilic drug.

In an embodiment, the water-dispersible single-chain dextran methacrylate or acrylate based nanoconjugate has a percentage of intra-molecular crosslinking from 3% to 45% molar % of the total amount of monomer units present in the dextran methacrylate or acrylate chain, particularly from 3% to 10% molar %. The percentage of intra-molecular crosslinking can be measured by any appropriate method known in the state of the art. In particular, the method used in the present invention is Nuclear Magnetic Resonance (NMR) spectroscopy; specially proton magnetic resonance spectroscopy.

As it is mentioned above, the nanoconjugates of the present invention have a high conjugation loading of the hydrophilic active ingredient, particularly a drug loading from 20% to 40% by weight of the drug in relation to the total weight of the nanoconjugate; particularly from 25 to 30%. The drug loading can be measured by any appropriate method known in the state of the art. In particular, the method used in the present invention can be nuclear Magnetic Resonance (NMR) spectroscopy; specially by proton magnetic resonance spectroscopy or ultraviolet spectroscopy for the conjugates of the present invention having a hydrolysable covalent bond; or alternatively by liquid chromatography-mass spectroscopy (LC-MS).

The term “arylene” refers to a bivalent aryl group, which contains one or more rings and having the number of carbon member atoms specified in the description or claims. The term “(C₅-C₈)aryl” refers to an aromatic known ring system comprising one or more rings and having from 5 to 8 ring members, wherein all the ring members comprise carbon atoms. Examples of (C₅-C₈)aryl include phenyl. The term “known ring system” as used herein refers to a ring system, which is chemically feasible and it is known in the art and so intends to exclude those ring systems that are not chemically possible.

In an embodiment, the water-dispersible single-chain dextran methacrylate or acrylate based nanoconjugate is one wherein [symbol] represents a non-covalent bond as defined above. As it is mentioned above, the nanoconjugate of the present invention which has the non-covalent bond between the drug and the nanoparticles are specially appropriate for achieving a rapid release of the hydrophilic active ingredient and therefore, having an immediate therapeutic effect.

In an embodiment, the water-dispersible single-chain dextran acrylate based nanoconjugate is one wherein: [symbol] represents a non-covalent bond; L₁ is —[(CH₂)ᵣO]_{q}—(CH₂)ᵣ; L₂ is —(CH₂)ᵥ—; R₁ is —CH₂—CH₂—; and R₂ is —CH₂—CH₂—S—(CH₂)ᵥ—COOH and q, r and v are as defined in the present invention having the following formula:

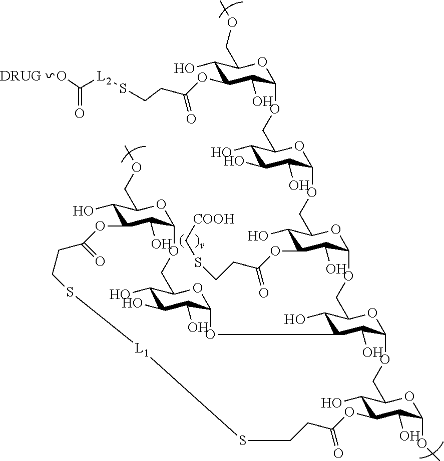

In an embodiment, the water-dispersible single-chain dextran methacrylate based nanoconjugate is one wherein: [symbol] represents a non-covalent bond; L₁ is —[(CH₂)ᵣO]_{q}—(CH₂)ᵣ; L₂ is —(CH₂)ᵥ; R₁ is —CH₂—CH(CH₃)—; and R₂ is —CH(CH₃)—CH₂—S—(CH₂)ᵥ—COOH; and q, r and v are as defined in the present invention having the following formula:

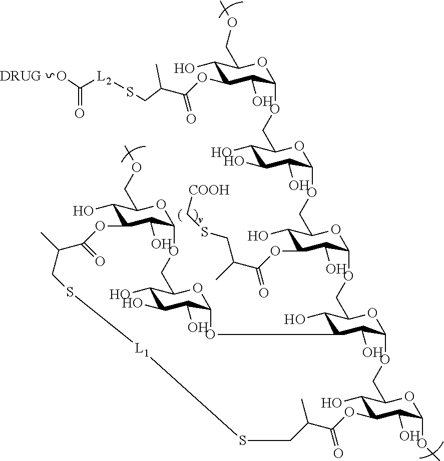

In an embodiment the water-dispersible single-chain dextran acrylate based nanoconjugate is one wherein: [symbol] represents a non-covalent bond; L₁ is —(CH₂)₂—O—(CH₂)₂—O—(CH₂)₂—; L₂ is —(CH₂)₂—; R₁ is —CH₂—CH₂—; and R₂ is-CH₂—CH₂—S—(CH₂)₂—COOH.

In an embodiment, the water-dispersible single-chain dextran methacrylate based nanoconjugate is one wherein: [symbol] represents a non-covalent bond; L₁ is —(CH₂)₂—O—(CH₂)₂—O—(CH₂)₂—; L₂ is —(CH₂)₂—; R₁ is —CH₂—CH(CH₃)—; and R₂ is —CH(CH₃)—CH₂—S—(CH₂)₂—COOH.

In an embodiment, the water-dispersible single-chain dextran methacrylate or acrylate based nanoconjugate is one wherein [symbol] represents a hydrolysable covalent bond as defined in the invention. As it is mentioned above, the nanoconjugate of the present invention which has the hydrolysable covalent bond between the drug and the nanoparticles are specially appropriate for achieving a controlled release of the hydrophilic active ingredient and therefore, being appropriate for reducing the number of intakes of the active ingredient. For the purpose of the invention, the term “hydrolysable bond” refers to a bond that can be cleaved in a reaction involving the addition of water, in such a way that the drug is released after delivery into the target site. In an embodiment, the hydrolysable covalent bond is selected from the group consisting of an ester, an amide, and a carbonate; particularly an ester. The appropriate cleavage reaction conditions of the drug can be readily determined by those skilled in the art according to the type of hydrolysable covalent bond present in the nanoconjugate.

In an embodiment, the water-dispersible single-chain dextran acrylate based nanoconjugate is one wherein: [symbol] represents a hydrolysable covalent bond; L₁ is —[(CH₂)ᵣO]_{q}—(CH₂)ᵣ; L₂ is —(C₅-C₆)arylene-NH—CO—(CH₂)ₚ—; R₁ is —CH₂—CH₂—; and R₂ is —CH(═CH₂) or —CH₂—CH₂—S—[(CH₂)_{w}—O]ₓ—(CH₂)_{w}—OH and p, q, r, x and w are as defined in the present invention.

In an embodiment, the water-dispersible single-chain dextran methacrylate based nanoconjugate is one wherein: [symbol] represents a hydrolysable covalent bond; L₁ is —[(CH₂)ᵣO]_{q}—(CH₂)ᵣ; L₂ is —(C₅-C₆)arylene-NH—CO—(CH₂)ₚ—; R₁ is —CH₂—CH(CH₃)—; and R₂ is C(═CH)(CH₃) or —CH(CH₃)—CH₂—S—[(CH₂)_{w}—O]ₓ—(CH₂)_{w}—OH, and p, q, r, x and w are as defined in the present invention.

In an embodiment, the water-dispersible single-chain dextran acrylate based nanoconjugate is one wherein: [symbol] represents a hydrolysable covalent bond; L₁ is —(CH₂)₂—O—(CH₂)₂—O—(CH₂)₂—; L₂ is

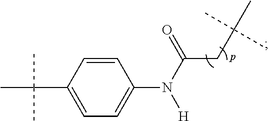

R₁ is —CH₂—CH₂—; and R₂ is —CH(═CH₂) or —CH₂—CH₂—S—[(CH₂)_{w}—O]ₓ—(CH₂)_{w}—OH and p, x and w are as defined in the present invention.

In an embodiment, the water-dispersible single-chain dextran methacrylate based nanoconjugate is one wherein: [symbol] represents a hydrolysable covalent bond; L₁ is —(CH₂)₂—O—(CH₂)₂—O—(CH₂)₂—; L₂ is

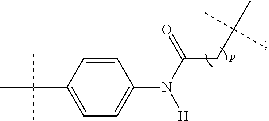

R₁ is —CH(CH₃)—CH₂—; and R₂ is —C(═CH₂)(CH₃) or —CH(CH₃)—CH₂—S—[(CH₂)_{w}—O]ₓ—(CH₂)_{w}—OH and p, x and w are as defined in the present invention.

In an embodiment, the water-dispersible single-chain dextran methacrylate based nanoconjugate is one wherein: [symbol] represents a hydrolysable covalent bond; L₁ is —(CH₂)₂—O—(CH₂)₂—O—(CH₂)₂—; L₂ is -phenylene-NH—CO—(CH₂)ₚ—; R₁ is —CH₂—CH(CH₃)—; and R₂ is —CH(CH₃)—CH₂—S—[(CH₂)ᵣ—O]_{q}—(CH₂)ᵣ—OH, and, q, r and p are as defined in the present invention.

In an embodiment, the water-dispersible single-chain dextran acrylate based nanoconjugate is one wherein: [symbol] represents a hydrolysable covalent bond; L₁ is —(CH₂)₂—O—(CH₂)₂—O—(CH₂)₂—; L₂ is

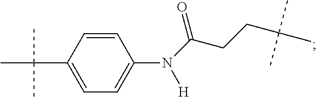

R₁ is-CH₂—CH₂—; and R₂ is-CH(═CH₂) or —CH₂—CH₂—S—[(CH₂)₂—O]₂—(CH₂)₂—OH.

In an embodiment, the water-dispersible single-chain dextran methacrylate based nanoconjugate is one wherein: [symbol] represents a hydrolysable covalent bond; L₁ is —(CH₂)₂—O—(CH₂)₂—O—(CH₂)₂—; L₂ is

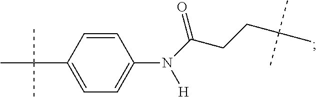

R₁ is —CH(CH₃)—CH₂— and R₂ is —C(═CH₂)(CH₃) or —CH(CH₃)—CH₂—S—[(CH₂)₂—O]₂—(CH₂)₂—OH.

In an embodiment, the water-dispersible single-chain dextran methacrylate based nanoconjugate is one of formula:

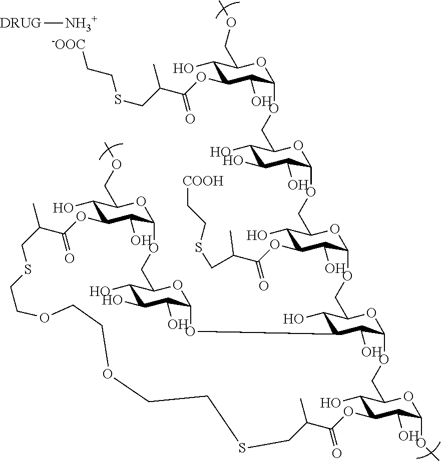

In an embodiment, the water-dispersible single-chain dextran acrylate based nanoconjugate is one of formula:

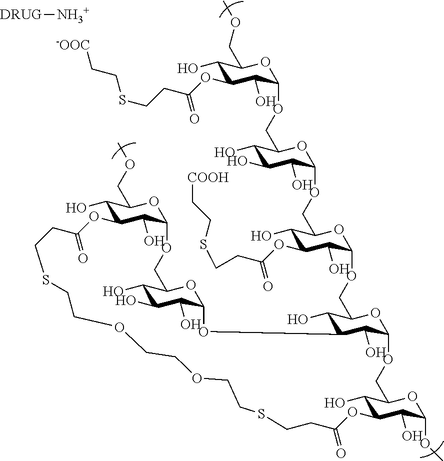

In an embodiment, the water-dispersible single-chain dextran methacrylate based nanoconjugate is one of formula:

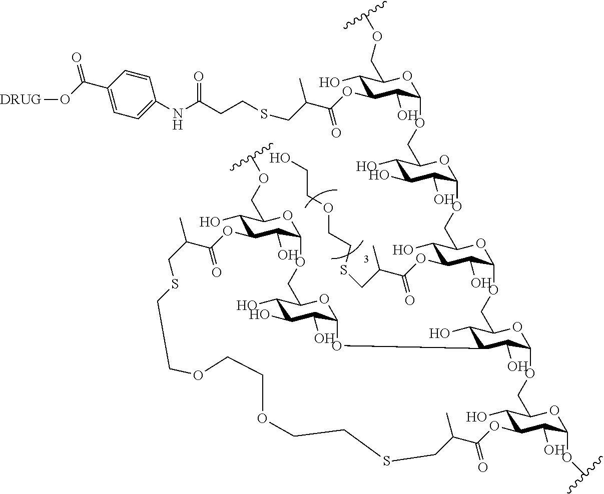

In an embodiment, the water-dispersible single-chain dextran acrylate based nanoconjugate is one of formula:

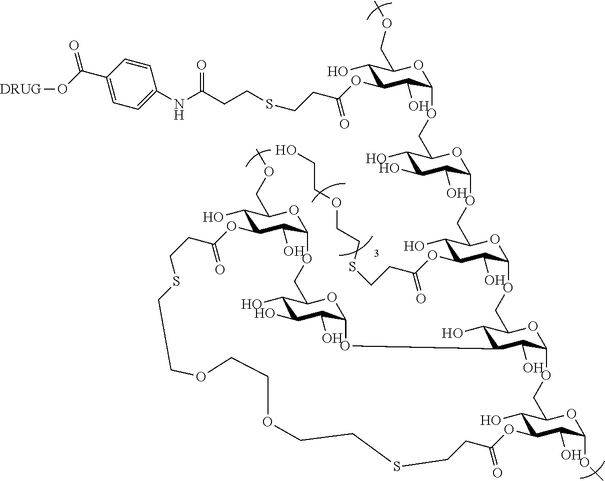

In an embodiment, the water-dispersible single-chain dextran methacrylate based nanoconjugate is one of formula:

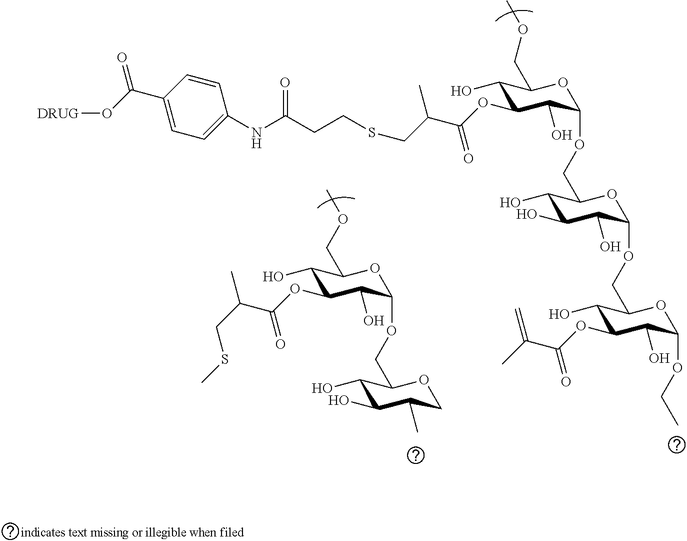

In an embodiment, the water-dispersible single-chain dextran acrylate based nanoconjugate is one of formula:

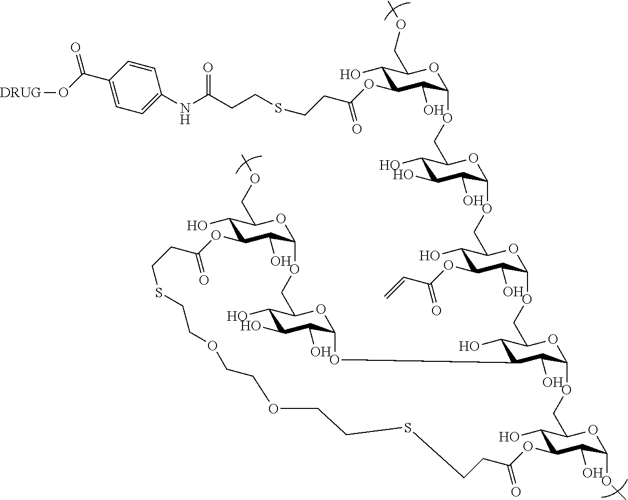

As it is mentioned above, the nanoconjugate of the present invention comprises one or more hydrophilic active ingredients. The terms “active ingredient”, “active substance”, “active pharmaceutical ingredient”, “drug” and “API” have the same meaning and are used interchangeably. They refer to a chemical substance that has a therapeutic effect in the field of pharmacy.

For the purpose of the present invention, the term “hydrophilic active ingredient” is to be understood as a drug that is charge-polarized and capable of hydrogen bonding, enabling it to dissolve more readily in water than in oil or other hydrophobic solvents. It is also known as “polar drug” and, both terms can be used interchangeably. On the contrary, the term “hydrophobic active ingredient” is to be understood as a drug, which tends to be non-polar and, thus, prefer other neutral molecules and non-polar solvents rather than water. Hydrophobic molecules in water often can form aggregates than can only be redispersed in water but not dissolved.

As it is well known for the skilled person in the art, a parameter useful to determine whether an active ingredient is hydrophilic or hydrophobic is determining its partition coefficient (P). The partition (P) coefficient is the ratio of concentrations of a particular compound in a mixture of two immiscible phases at equilibrium. Normally one of the solvents chosen is water while the second is hydrophobic such as octanol. Hydrophobic active ingredients have high octanol/water partition coefficients, and hydrophilic active ingredients have low octanol/water partition coefficients. The log P value is also known as a measure of lipophilicity/hydrophilicity. The logarithm of the ratio of the concentrations of the un-ionized solute in the solvents, at a specific pH, is called log P: The log P value is also known as a measure of lipophilicity:

$$log {P}_{oct / wat} = log ( \frac{{[ solute ]}_{octanol}}{{[ solute ]}_{water}^{un \text{-} ionized}} )$$

wherein the “solute” is the active ingredient.

Therefore, an active ingredient is “hydrophilic” wherein the log P values is lower than 0.1 measured at a pH from 6 to 8. On the contrary, an active ingredient is “lipophilic” wherein the log P value is equal or higher than 0.1 measured at pH from 6 to 8.

In an embodiment, the water-dispersible single-chain dextran methacrylate or acrylate based nanoconjugate of the present invention is one wherein the hydrophilic active ingredient has a log P value from −3 to −9 measured at pH from 6 to 8.

The log P values of the most common active ingredients are disclosed in the state of the art. In particular, the log P values of the corresponding pharmaceutically acceptable salt of most common active ingredients are also disclosed in the state of the art. In fact, the log P of a pharmaceutically acceptable salt of a hydrophilic active ingredient corresponds to the log P value of the hydrophilic active ingredient in free form.

In an embodiment, the water-dispersible single-chain dextran methacrylate or acrylate based nanoconjugate of the present invention is one wherein the hydrophilic active ingredient is a hydrophilic antibiotic. The term “antibiotic” refers to a type of antimicrobial substance active against bacteria, which is nowadays the most important type of antibacterial agent for fighting bacterial infections. In an embodiment, the water-dispersible single-chain dextran methacrylate or acrylate based nanoconjugate of the present invention is one wherein the hydrophilic active ingredient is a hydrophilic antibiotic selected from the group consisting of aminoglycoside, polymyxin, glycopeptide and antimicrobial peptides.

In an embodiment, the water-dispersible single-chain dextran methacrylate or acrylate based nanoconjugate of the present invention is one wherein the hydrophilic active ingredient is a hydrophilic aminoglycoside type antibiotic selected from the group consisting of gentamicin, tobramycin, amikacin, kanamycin, neomycin, netilmicin, paromomycin and streptomycin; particularly tobramycin and gentamycin; more particularly tobramycin.

In an embodiment, the water-dispersible single-chain dextran methacrylate or acrylate based nanoconjugate of the present invention is one wherein the hydrophilic active ingredient is a hydrophilic polymyxin type antibiotic selected from the group consisting of colistin, polymixin B and bacitracin; particularly colistin.

In an embodiment, the water-dispersible single-chain dextran methacrylate or acrylate based nanoconjugate of the present invention is one wherein the hydrophilic active ingredient is a hydrophilic glycopeptide type antibiotic selected from the group consisting of vancomycin and telavancin.

In an embodiment, the water-dispersible single-chain dextran methacrylate or acrylate based nanoconjugate of the present invention is one wherein the hydrophilic active ingredient is a hydrophilic antimicrobial peptide selected from the group consisting of LL37 and 1WCO.

In an embodiment, the water-dispersible single-chain dextran methacrylate based nanoconjugate of the present invention is one of formula (I-1)

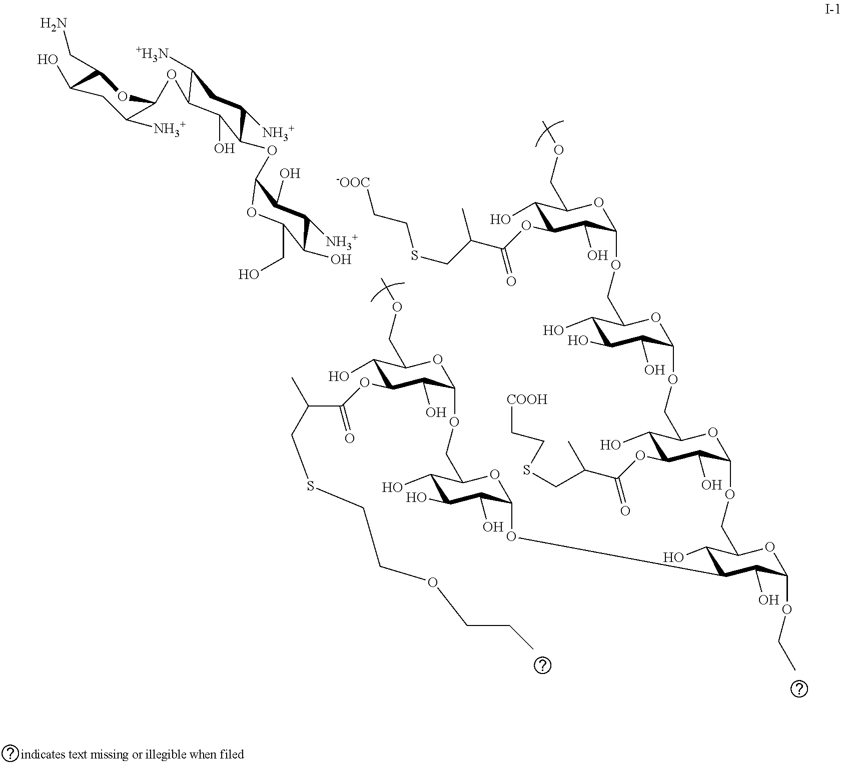

In an embodiment, the water-dispersible single-chain dextran acrylate based nanoconjugate of the present invention is one of formula (I-2)

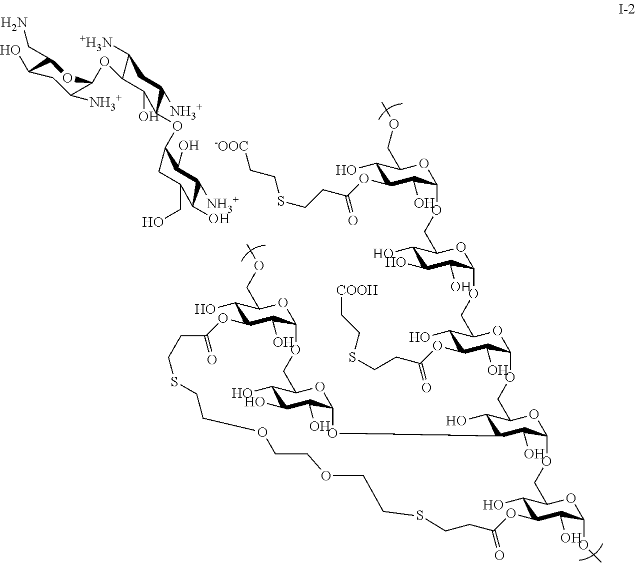

In an embodiment, the water-dispersible single-chain dextran methacrylate based nanoconjugate of the present invention is one of formula (I-3)

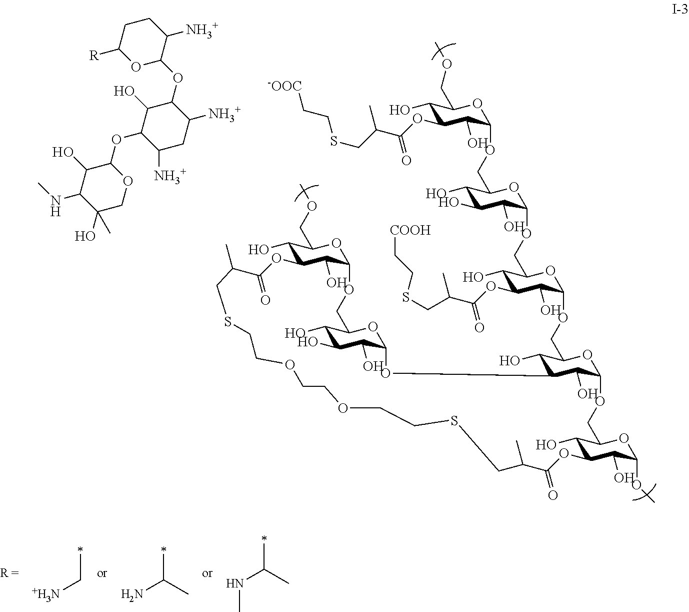

In an embodiment, the water-dispersible single-chain dextran acrylate based nanoconjugate of the present invention is one of formula (I-4)

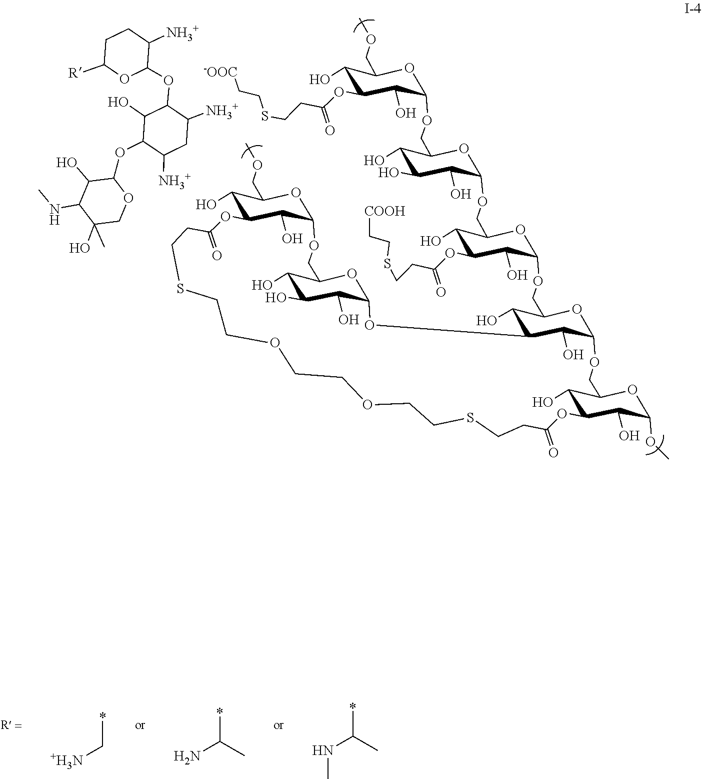

In an embodiment, the water-dispersible single-chain dextran methacrylate based nanoconjugate of the present invention is one of formula (I-5)

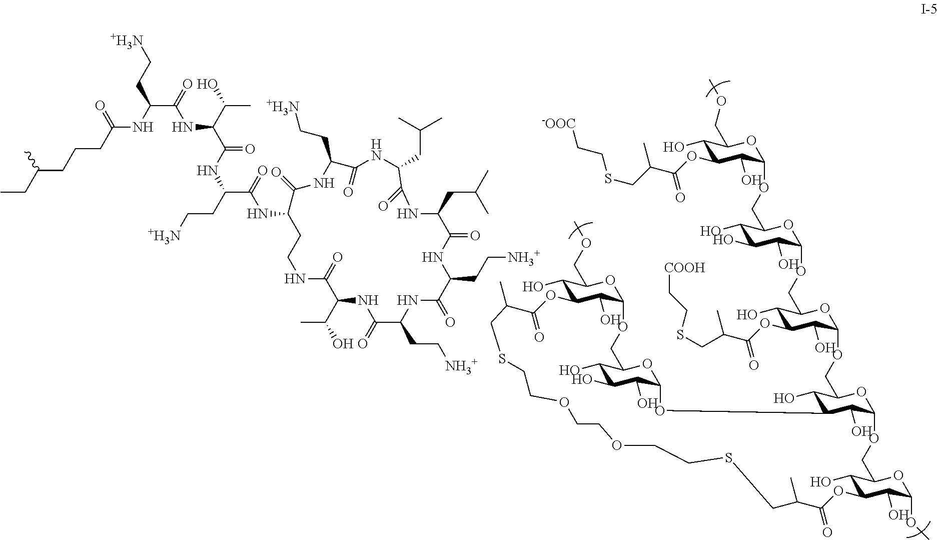

In an embodiment, the water-dispersible single-chain dextran acrylate based nanoconjugate of the present invention is one of formula (I-6)

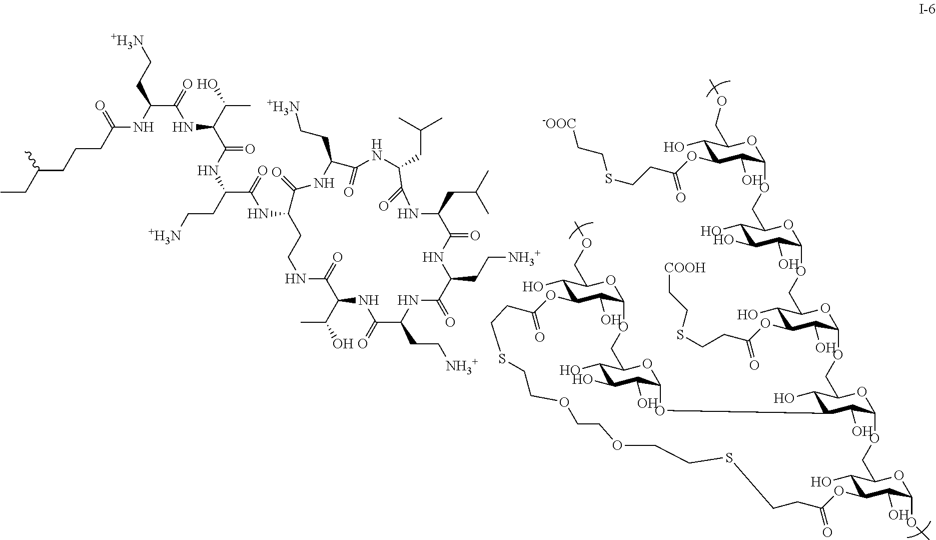

In an embodiment, the water-dispersible single-chain dextran methacrylate based nanoconjugate of the present invention is one of formula (I-7)

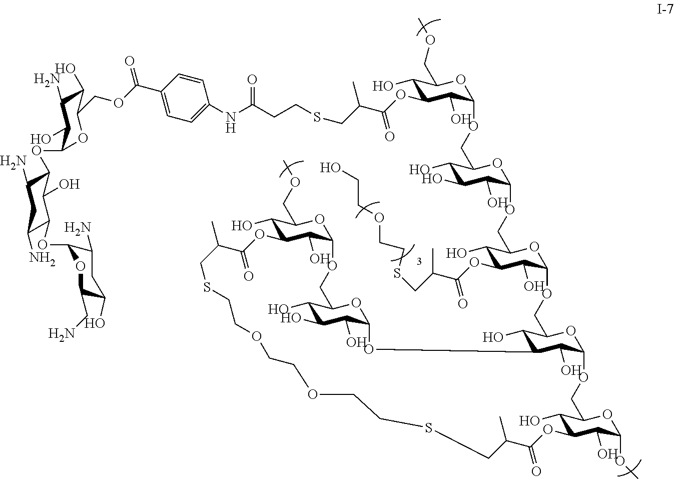

In an embodiment, the water-dispersible single-chain dextran methacrylate based nanoconjugate of the present invention is one of formula (I-8)

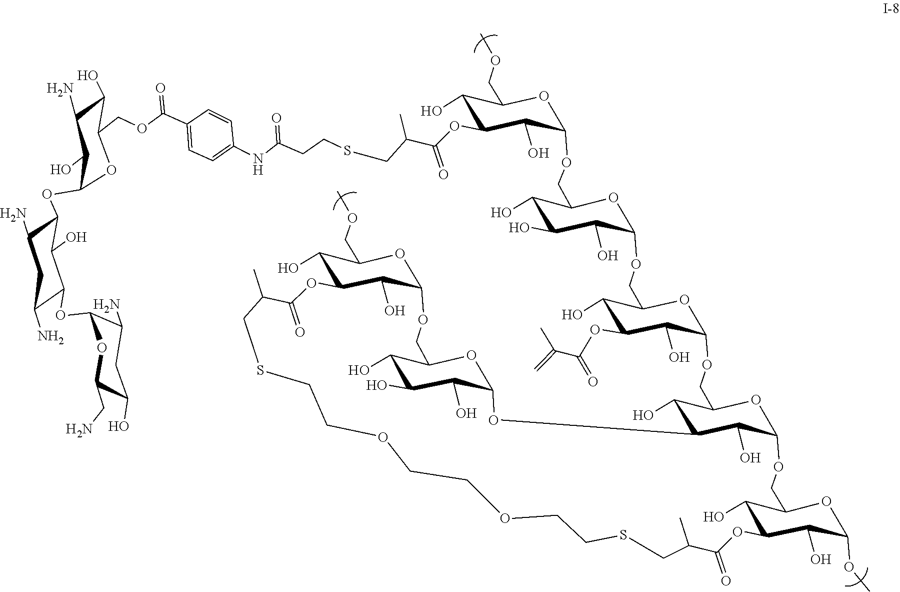

In an embodiment, the water-dispersible single-chain dextran acrylate based nanoconjugate of the present invention is one of formula (I-9)

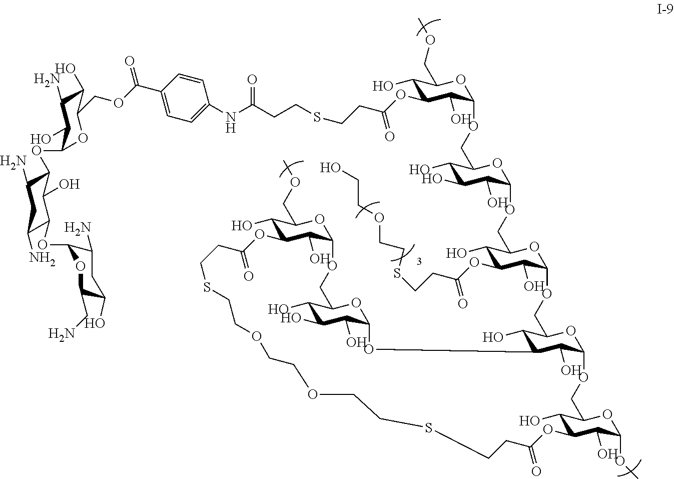

In an embodiment, the water-dispersible single-chain dextran acrylate based nanoconjugate of the present invention is one of formula (I-10)

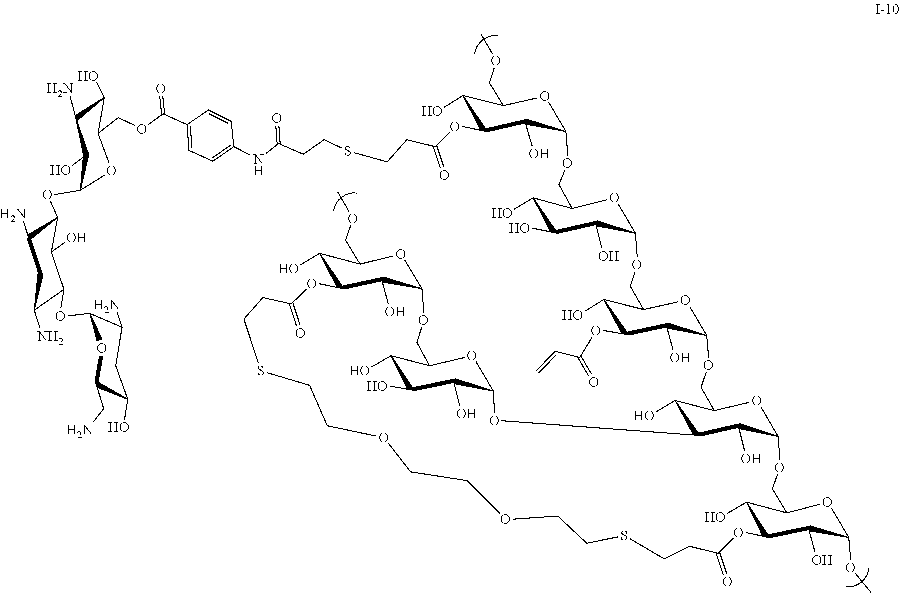

In an embodiment, the water-dispersible single-chain dextran methacrylate based nanoconjugate of the present invention is one of formula (I-11)

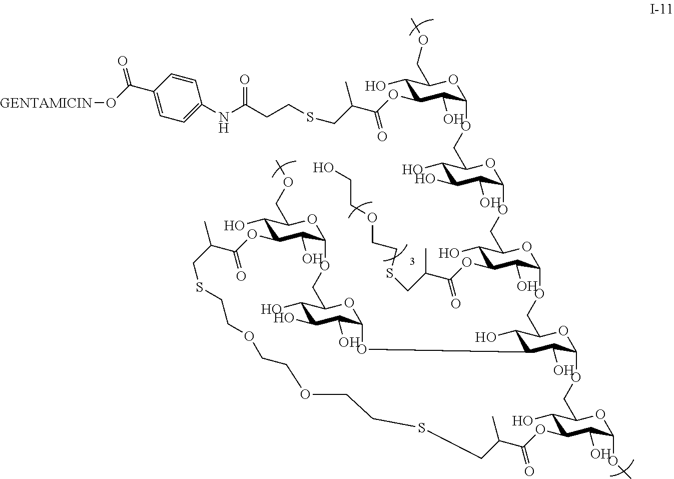

In an embodiment, the water-dispersible single-chain dextran acrylate based nanoconjugate of the present invention is one of formula (I-12)

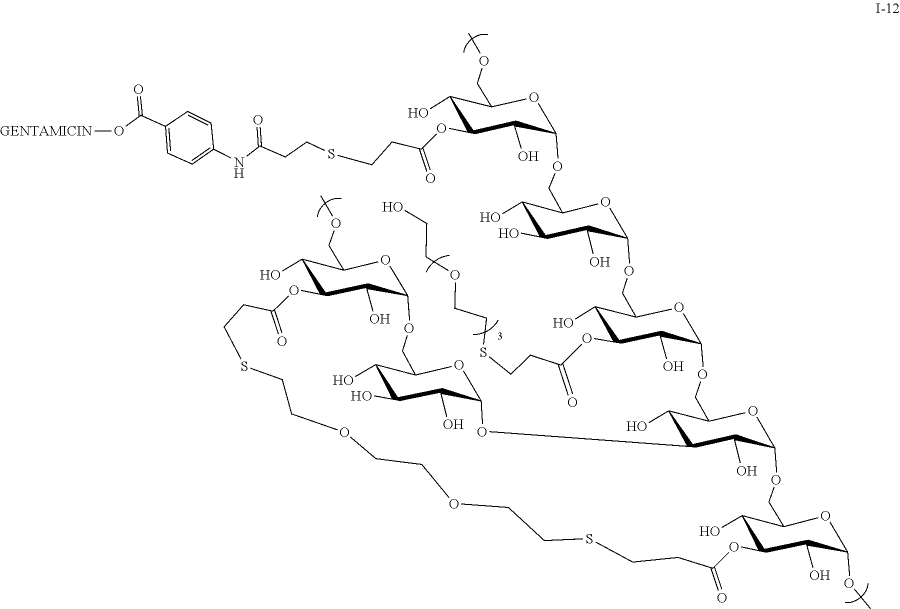

In an embodiment, the water-dispersible single-chain dextran methacrylate based nanoconjugate of the present invention is one of formula (I-13)

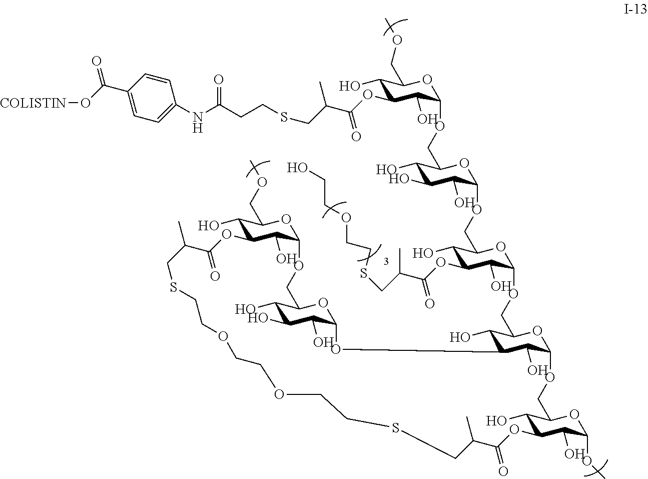

In an embodiment, the water-dispersible single-chain dextran acrylate based nanoconjugate of the present invention is one of formula (I-14)

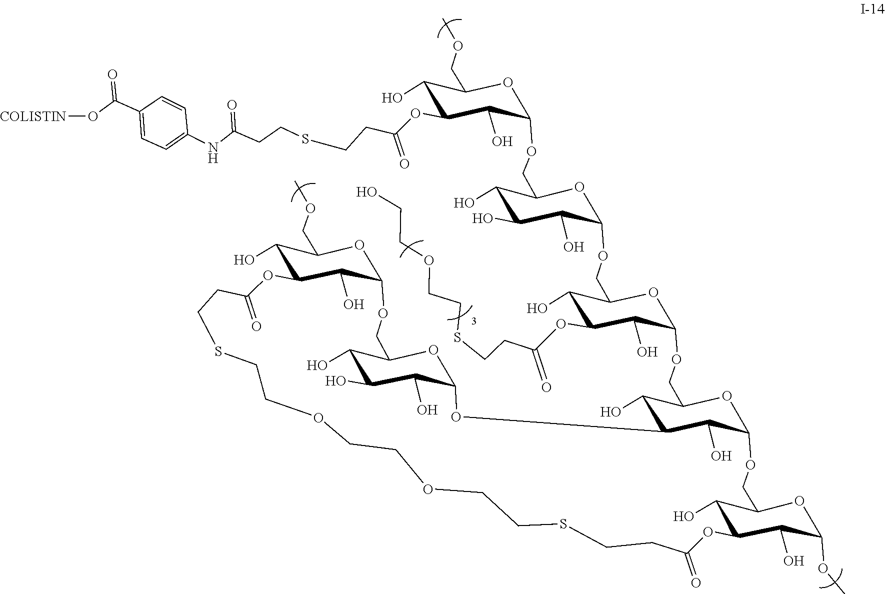

The second aspect of the invention relates to a process for the preparation of the water-dispersible single-chain dextran methacrylate or acrylate based nanoconjugate of the first aspect of the invention.

In an embodiment, the process for the preparation of the water-dispersible single-chain dextran methacrylate or acrylate based nanoconjugate of the invention wherein [symbol] represents a hydrolysable covalent bond, then the process comprises:

a) reacting a single-chain dextran methacrylate or acrylate based nanoparticle having a particle size from 3 to 50 nm, a polydispersion of the nanoparticle equal to the polydispersion of dextran ±20%; and an intra-molecular crosslinking from 3 to 45 molar % of the total amount of monomer units present in the dextran methacrylate or acrylate chain with a compound of formula

DRUG-O—CO-L₂-SH

at a pH from 7.5 to 10 and at a temperature form 20° C. to 25° C. to obtain a water-dispersible single-chain dextran methacrylate or acrylate based nanoconjugate as defined in the present invention, wherein R₂ is selected from the group consisting of —CH(═CH₂) and —C(═CH₂)(CH₃).

In an embodiment, the process for the preparation of the water-dispersible single-chain dextran methacrylate or acrylate based nanoconjugate of the invention wherein [symbol] represents a hydrolysable covalent bond, then the process further comprises:

b) reacting the water-dispersible single-chain dextran methacrylate or acrylate based nanoconjugate obtained in claim a) with a compound of SH—[(CH₂)_{w}—O]ₓ—(CH₂)_{w}—OH under such reaction conditions to obtain a water-dispersible single-chain dextran methacrylate or acrylate based nanoconjugate as defined in the present invention, wherein R₂ is selected from the group consisting of —CH₂—CH₂—S—[(CH₂)_{w}—O]ₓ—(CH₂)_{w}—OH, and —CH(CH₃)—CH₂—S—[(CH₂)_{w}—O]ₓ—(CH₂)_{w}—OH.

Thus, the process for the preparation of the water-dispersible single-chain dextran methacrylate or acrylate based nanoconjugate of the invention wherein [symbol] represents a hydrolysable covalent bond and R₂ is selected from the group consisting of —CH₂—CH₂—S—[(CH₂)_{w}—O]ₓ—(CH₂)_{w}—OH, and —CH(CH₃)—CH₂—S—[(CH₂)_{w}—O]ₓ—(CH₂)_{w}—OH comprises:

a) reacting a single-chain dextran methacrylate or acrylate based nanoparticle having a particle size from 3 to 50 nm, a polydispersion of the nanoparticle equal to the polydispersion of dextran ±20%; and an intra-molecular crosslinking from 3 to 45 molar % of the total amount of monomer units present in the dextran methacrylate or acrylate chain with a compound of formula

DRUG-O—CO-L₂-SH

at a pH from 7.5 to 10 and at a temperature form 20° C. to 25° C. to obtain a water-dispersible single-chain dextran methacrylate or acrylate based nanoconjugate as defined in the present invention, wherein R₂ is selected from the group consisting of —CH(═CH₂) and —C(═CH₂)(CH₃), and

b) reacting the water-dispersible single-chain dextran methacrylate or acrylate based nanoconjugate obtained in claim a) with a compound of formula SH—[(CH₂)_{w}—O]ₓ—(CH₂)_{w}—OH under such reaction conditions to obtain a water-dispersible single-chain dextran methacrylate or acrylate based nanoconjugate as defined in the present invention, wherein R₂ is selected from the group consisting of —CH₂—CH₂—S—[(CH₂)_{w}—O]ₓ—(CH₂)_{w}—OH, and —CH(CH₃)—CH₂—S—[(CH₂)_{w}—O]ₓ—(CH₂)_{w}—OH.

As it is mentioned above, step (a) of the process of the invention is performed at a pH from 7.5 to 10. In an embodiment, step (a) of the process of the invention is performed at a pH from 8 to 10. In an embodiment, the step (a) of the process of the invention is performed at a pH from 7.5 to 9. In an embodiment, the step (a) of the process of the invention is performed at a pH from 8 to 9.

Further, as it is also mentioned above, step (a) of the process of the invention is performed at room temperature. The term “room temperature” refers to a temperature of the environment, without heating or cooling, and is generally a temperature from 20° C. to 25° C.

In an embodiment, wherein the process comprises performing step (b), then step (b) is performed at a pH from 7.5 to 10. In an embodiment, step (b) of the process of the invention is performed at a pH from 8 to 10. In an embodiment, the step (b) of the process of the invention is performed at a pH from 7.5 to 9. In an embodiment, the step (b) of the process of the invention is performed at a pH from 8 to 9.

In an embodiment, wherein the process comprises performing step (b), then step (b) is performed at room temperature.

In an embodiment, the process for the preparation of the water-dispersible single-chain dextran methacrylate or acrylate based nanoconjugate of the invention wherein [symbol] represents a non-covalent bond, then the process comprises:

[symbol]

c) reacting a single-chain dextran methacrylate or acrylate based nanoparticle having a particle size from 3 to 50 nm, a polydispersion of the nanoparticle equal to the polydispersion of dextran ±20%; and an intra-molecular crosslinking from 3 to 45 molar % of the total amount of monomer units present in the dextran methacrylate or acrylate chain with a compound of formula

HS-L₂-COOH

and d) contacting the single-chain dextran methacrylate or acrylate based nanoparticle obtained in step c) with the DRUG and adjusting the pH to 7 by the addition of an acid to obtain the water-dispersible single-chain dextran methacrylate or acrylate based nanoconjugate; wherein R₂ is selected from the group consisting of —CH₂—CH₂—S—(CH₂)ᵥ—COOH and —CH(CH₃)—CH₂—S—(CH₂)ᵥ—COOH.

In an embodiment, the process for the preparation of the water-dispersible single-chain dextran methacrylate or acrylate based nanoconjugate of the invention wherein [symbol] represents a non-covalent bond, then the process further comprises:

Thus, the process for the preparation of the water-dispersible single-chain dextran methacrylate or acrylate based nanoconjugate of the invention wherein [symbol] represents a non-covalent bond, wherein R₂ is selected from the group consisting of —CH₂—CH₂—S—(CH₂)ᵥ—COOH and —CH(CH₃)—CH₂—S—(CH₂)ᵥ—COOH comprises:

c) reacting a single-chain dextran methacrylate or acrylate based nanoparticle having a particle size from 3 to 50 nm, a polydispersion of the nanoparticle equal to the polydispersion of dextran ±20%; and an intra-molecular crosslinking from 3 to 45 molar % of the total amount of monomer units present in the dextran methacrylate or acrylate chain with a compound of formula

HS-L₂-COOH

and d) contacting the single-chain dextran methacrylate or acrylate based nanoparticle obtained in step c) with the DRUG and adjusting the pH to 7 by the addition of an acid to obtain the water-dispersible single-chain dextran methacrylate or acrylate based nanoconjugate; wherein R₂ is selected from the group consisting of —CH₂—CH₂—S—(CH₂)ᵥ—COOH and —CH(CH₃)—CH₂—S—(CH₂)ᵥ—COOH.

In an embodiment, step c) of the process of the invention is performed by reacting a methacrylate or acrylate based nanoparticle with an intra-molecular crosslinking from 2 to 10 molar % of the total amount of monomer units present in the dextran chain. In an embodiment, step c) of the process of the invention is performed by reacting a methacrylate or acrylate based nanoparticle with an intra-molecular crosslinking from 4 to 7 molar % of the total amount of monomer units present in the dextran chain.

In an embodiment, the processes as defined above for the preparation of the nanoconjugate of the present invention further comprise an additional step of purifying the water-dispersible single-chain dextran methacrylate or acrylate based nanoconjugate. Any method appropriate for purifying nanoconjugates disclosed in the state of the art can be used. In particular, the purifying step is selected from the group consisting of dialysis, centrifuge filtration, column separation techniques, and precipitation; preferably, the purifying step is carried out by dialysis.

In an embodiment, the processes as defined above for the preparation of the nanoconjugate of the present invention further comprise an additional step of drying the water-dispersible single-chain dextran methacrylate or acrylate based nanoconjugate. Any method appropriate for drying nanoconjugates disclosed in the state of the art can be used. In particular, the drying step is selected from the group consisting of spray drying, and freeze-drying. In a preferred embodiment, the drying step carried out is freeze-drying.

In an embodiment, the processes as defined above for the preparation of the nanoconjugate of the present invention further comprise additional steps, wherein the additional steps of purifying and drying the nanoconjugate obtained following the processes as defined above.

In an embodiment, the processes as defined above further comprises a previous step of preparing the single-chain dextran methacrylate or acrylate based nanoparticle having a particle size from 3 to 50 nm, a polydispersion of the nanoparticle equal to the polydispersion of dextran ±20%; and an intra-molecular crosslinking from 3 to 45 molar % of the total amount of monomer units present in the dextran chain.

In an embodiment, the processes as defined above further comprises a previous step of preparing the single-chain dextran methacrylate or acrylate based nanoparticle by a process which comprises cross-linking a dextran methacrylate or acrylate having a solubility equal to or higher than 100 mg per litre of water, and an amount of methacrylate or acrylate groups from 5 to 60 molar % of the total amount of monomer units present in the dextran chain; with a homobifunctional crosslinking agent having crosslinkable groups, at a temperature from 20 to 25° C. in the absence of a catalyst.

In an embodiment, the processes as defined above further comprises a previous step of preparing the single-chain dextran methacrylate or acrylate based nanoparticle by an alternative process which comprises: (e1) adding an aqueous solution of dextran methacrylate or acrylate having an amount of methacrylate or acrylate groups from 5 to 60 molar % of the total amount of monomer units present in the dextran chain to an aqueous solution of the homobifunctional crosslinking agent at an addition rate that allows having during the addition of dextran methacrylate or acrylate a concentration of the crosslinkable groups of the dextran methacrylate or acrylate in the solution of the homobifunctional crosslinking agent from 5×10⁻³ to 10⁻⁷ molar, at a temperature from 20 to 25° C. in the absence of a catalyst.

In an embodiment, the process for the preparation of the water-dispersible single-chain dextran methacrylate or acrylate based nanoconjugate as defined above further comprises a previous step of preparing the single-chain dextran methacrylate or acrylate based nanoparticle by an alternative process which comprises: (e2) adding an aqueous solution of the homobifunctional crosslinking agent to an aqueous solution of the dextran methacrylate or acrylate having an amount of methacrylate or acrylate groups from 5 to 60 molar % of the total amount of monomer units present in the dextran chain at an addition rate that allows having during the addition of the homobifunctional crosslinking agent a concentration of the homobifunctional crosslinking agent in the solution of dextran methacrylate or acrylate from 5×10⁻³ to 10⁻⁷ molar, at a temperature from 20 to 25° C. in the absence of a catalyst.

In an embodiment, the process for the preparation of the water-dispersible single-chain dextran methacrylate or acrylate based nanoconjugate as defined above further comprises a previous step of preparing the single-chain dextran methacrylate or acrylate based nanoparticle by an alternative process which comprises: (e3) mixing a solution of the homobifunctional crosslinking agent and an aqueous solution of the dextran methacrylate or acrylate having an amount of methacrylate or acrylate groups from 5 to 60 molar % of the total amount of monomer units present in the dextran chain having a concentration of the homobifunctional crosslinking agent in the mixture from 10⁻⁷ to 5·10⁻³ molar, at a temperature from 20 to 25° C. in the absence of a catalyst.

In an embodiment, the processes for the preparation of the water-dispersible single-chain dextran methacrylate or acrylate based nanoparticle as defined above further comprise an additional purifying step after steps (e1), (e2) or (e3). Any method appropriate for purifying nanoparticles disclosed in the state of the art can be used. In particular, the purifying step is selected from the group consisting of dialysis, centrifuge filtration, column separation techniques, and precipitation; preferably, the purifying step is carried out by dialysis.

In an embodiment, the processes for the preparation of the water-dispersible single-chain dextran methacrylate or acrylate based nanoparticle as defined above further comprise an additional drying step after steps (e1), (e2) or (e3). Any method appropriate for drying nanoparticles disclosed in the state of the art can be used. In particular, the drying step is selected from the group consisting of spray drying, and freeze-drying. In a preferred embodiment, the drying step carried out is freeze-drying.

The term hydrophilic active ingredient (also called DRUG) used as a starting material for the preparation of the water-dispersible single-chain dextran methacrylate or acrylate based nanoconjugates of the present invention encompasses both the hydrophilic active ingredient in free form and also its pharmaceutically acceptable salts thereof. The term “pharmaceutically acceptable salt” used herein encompasses any salt formed from pharmaceutically acceptable non-toxic acids including inorganic or organic acids. There is no limitation regarding the salts, except that they must be pharmaceutically acceptable because they are used for therapeutic purposes. Most of these pharmaceutically acceptable salts are commercially available. If not, these salts can be prepared following the processes disclosed in the state of the art, which involves starting from pharmaceutically acceptable non-toxic acids, including inorganic and organic acids. Such acids include for instance acetic, benzenesulfonic, benzoic, camphorsulfonic, citric, ethansulfonic, fumaric, gluconic, glutamic, hydrobromic, hydrochloric, lactic, maleic, malic, mandelic, methanesulfonic, phosphoric, succinic, sulfuric, tartaric, p-toluensulfonic acid and hydrochloride.

The water-dispersible single-chain dextran methacrylate or acrylate based nanoconjugate obtainable by a process as defined above is also part of the invention. For the purposes of the invention the expressions “obtainable”, “obtained” and equivalent expressions are used interchangeably, and in any case, the expression “obtainable” encompasses the expression “obtained”.

For the purpose of the invention, when the water-dispersible single-chain dextran nanoconjugate is a methacrylate based nanoconjugate then the dextran used as starting material is functionalised with methacrylate groups; and alternatively, when the water-dispersible single-chain dextran nanoconjugate is an acrylate based nanoconjugate then the dextran used as starting material is functionalised with acrylate groups.

In an embodiment, the dextran with protected methacrylate or acrylate groups has a polydispersion of the particle equal to the polydispersion of the dextran methacrylate or acrylate ±20%; particularly ±15%; more particularly ±10%.

The third aspect of the invention relates to a pharmaceutical composition comprising a therapeutically effective amount of the water-dispersible nanoconjugate of the present invention, together with one or more appropriate pharmaceutically acceptable excipients or carriers.

The expression “therapeutically effective amount” as used herein, refers to the amount of the nanoconjugate of the present invention that, when administered, is sufficient to prevent development of, or alleviate to some extent, one or more of the symptoms of the disease which is addressed. For the purpose of the invention the term “therapeutically effective amount” refers to the amount of the nanoconjugate which provides a therapeutic effect after its application, which means the amount of the nanoconjugate that after administration provides a therapeutically effective amount of free hydrophilic active ingredient. The particular dose of nanoconjugate administered according to this invention will of course be determined by the particular circumstances surrounding the case, including the hydrophilic active ingredient, the route of administration, the particular condition being treated, and the similar considerations.

The term “pharmaceutically acceptable” refers to those excipients or carriers suitable for use in the pharmaceutical technology for preparing compositions with medical use.

The compositions of the invention can be formulated in several forms that include, but are not limited to, oral, rectal, dermal, mucosal, inhalable, transdermal and parenteral compositions.

In an embodiment, the pharmaceutical composition is a inhalable composition comprising “inhalable” water-dispersible single-chain dextran methacrylate or acrylate based nanoconjugate of the present invention, together with one or more “appropriate coarse excipients or carriers”. The term “inhalable nanoconjugate” refers to the nanoconjugates of the present invention that are suitable for pulmonary administration, capable of being transported deep into the branches of the lungs and being able to develop a local or a systemic activity, optionally through the alveoli. Generally, the particle size of an “inhalable particle” is up to 10 μm, more preferably from 0.5 to 10 μm, more preferably 0.5-6 μm. The term “coarse excipients or carriers” refers to those excipients or carriers having a particle size which makes them not inhalable. Generally, the particle size of a “coarse excipient or carrier” is from 15 to 250 μm. Inhalable nanoconjugates can be dispersed and inhaled by means of an inhaler. Suitable inhalers for being used with the nanoconjugates of the present invention includes, without limitation, metered dose inhalers (MDI) and dry powder inhalers or alternatively nebulizers.

The compositions of the present invention can be prepared according to methods well known in the state of the art. The appropriate excipients and/or carriers, and their amounts, can readily be determined by those skilled in the art according to the type of formulation being prepared.

As it is mentioned above, the nanoconjugates of the present invention comprises one or more hydrophilic active ingredients. Thus, it is also part of the invention the water-dispersible dextran methacrylate or acrylate based conjugate as defined in the present invention for use in therapy. It also relates to a method for the prophylaxis and/or treatment of a disease which comprises administering to mammals in need of such treatment an effective amount of the water-dispersible conjugate of the present invention, together with one or more appropriate pharmaceutically acceptable excipients or carriers.

In an embodiment, the water-dispersible conjugate is that wherein the hydrophilic active agent is a hydrophilic antibiotic as defined above. Then, in an embodiment, the water-dispersible nanoconjugate of the present invention is useful in the treatment of bacterial infection. Thus, another aspect of the present invention is the water-dispersible which comprises a hydrophilic antibiotic as defined above, for use in the prophylaxis and/or treatment of a disease or condition which transcur through a bacterial infection. This aspect could be also formulated as the use of the water-dispersible conjugate which comprises a hydrophilic antibiotic as defined above for the preparation of a medicament for the prophylaxis and/or treatment of a disease or condition which transcur through a bacterial infection. It also relates to a method for the prophylaxis and/or treatment of a mammal suffering or is susceptible to suffer from a disease or condition which transcur through a bacterial infection, the method comprises administering to said mammal an effective amount of the water-dispersible conjugate of the present invention which comprises a hydrophilic antibiotic.

In an embodiment, the water-dispersible nanoconjugate of the present invention which comprises a hydrophilic antibiotic is useful in the treatment of bacterial infection which is a lower respiratory track bacterial infection selected from patients with lung infections who suffer from the group consisting of chronic obstructive pulmonary diseases, cystic fibrosis, bronchoectasia, and hospital induced pneumonia. Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

The following abbreviations are used in the below examples:

PEG: polyethylenglycol

SEC: size exclusion chromatography

MWCO: molecular weight cut-off

General Considerations

Materials

Glycidyl methacrylate (GMA) (97%), dimethyl sulfoxide (DMSO) (98%), 3-mercaptopropionic acid (≥99%), 4-(dimethylamino)pyridine (DMAP)(96%), 2,2′-(ethylenedioxy)diethanethiol [3,6-dioxa-1,8-octane-dithiol (DODT)] (95%), 2-{2-[2-(2-Mercaptoethoxy)ethoxy]ethoxy}ethanol (MEEE, 97%), vinyl acrylate (VA) anddextran 70 kDa (DXT70) were purchased from Aldrich. Phosphate-buffered saline (PBS) was purchased from Scharlau. Dextran pharmaceutical grade (Dextran 40 Ultra—DXT40) was purchased from Pharmacosmos (Denmark) and Dextran 20 (DXT20) was purchased from Alfa Aesar.

Colistin sulfate was purchased from Cayman, gentamycin sulfate was purchased from Aldrich and tobramycin was purchased from Acros Organics.

Characterization Methods

Dynamic Light Scattering (DLS): DLS analyses were conducted using a Zetasizer Nano ZS, ZEN3600 Model (Malvern Instruments Ltd). All measurements were performed in disposable sizing cuvettes at a laser wavelength of 633 nm and a scattering angle of 173°, while the zeta-potential measurements were performed in disposable zeta potential cells (pH 7.4, 25° C.). Before the measurement, dextran methacrylate or acrylate based nanoparticles and nanoconjugates were dispersed in saline solution (0.9 wt % NaCl for size measurements and 1 mM NaCl for zeta-potential measurements) at a concentration of 1 mg/mL. Each measurement was repeated for three runs per sample at 25° C. PDI (size) is the value obtained from the square of the standard deviation divided by the mean diameter. The polydispersity of the nanoparticles size (PDI (size)) refers to the broadness of the size distribution obtained by Dynamic Light Scattering (DLS).

Nuclear magnetic resonance (¹H NMR): NMR spectra were recorded on a Bruker AVANCE III spectrometer at 500 MHz and 25° C. Chemical shifts (6) are given in ppm relative to the residual signal of the solvent. Splitting patterns: b, broad; s, singlet; d, doublet; t, triplet; q, quartet; m, multiplet.

Gel Permeation chromatography (GPC): The weight-average molecular weight (Mw), number-average molecular weight (Mn) and polydispersion of the polymer or SCNP (Mw/Mn) were measured at 40° C. on an Agilent GPC-50 system equipped with 2×PL-Aquagel Mixed-OH, Guard-Aquagel-OH columns and a differential refractive index (RI) detector. 0.3 M NaNO₃, 0.01 M NaH₂PO₄, pH 7 was used as eluent at a flow rate of 1 mL/min. The system was calibrated using polyethylene oxide (PEO) standards.

Efficiency in Vitro Tests with Klebsiella pneumoniae

Efficiency screening of nanoconjugates was performed using the microdilution method according to CLSI guideline (M07-A8) to determine the minimum inhibitory concentration (MIC) of the nanoconjugates versus Klebsiella pneumoniae—ESBL. The MIC assay was performed for dextran methacrylate or acrylate based nanoconjugates of the present invention. As positive controls, the free antibiotic was used. As negative controls, the respective unloaded nanocarriers (nanoparticles prior to the linkage of the hydrophilic active ingredient) were used.

The MIC assay for efficiency screening of the above listed nanoconjugates and nanoparticles was performed using three different K. pneumoniae strains: a) wild type (WT), b) beta-lactam resistant (ESBL), and c) ESBL and carbapenem resistant (ESBL-KPC) (received from partner EMC). To perform the MIC assay, the nanoconjugates and nanoparticles, blank controls and free AMP or MEM were titrated in 1:2 dilution series and 50 μl per well were added to 96 well microtiter plates. For each assay, the test organisms were grown from stock aliquots stored at −80° C. by overnight culture in tryptic soy bouillon at 37° C. Then, the K. pneumoniae suspensions were harvested and adjusted in cation-adjusted Mueller-Hinton broth (CAMHB) via turbidity measurement to a final germ number of 106 CFU/ml. 50 μl per well were added to the test items (nanoconjugates and nanoparticles, blanks or free AMP/MEM) or growth control wells without any test item. A contamination control was performed by wells with assay medium only and no addition of test germs. The microtiter plates were sealed with parafilm to prevent evaporation and incubated at 35±2° C. for 16-20 h. Antimicrobial efficacy of the preservative systems was evaluated by assessment of microbial growth by macroscopic turbidity control. The minimal inhibitory concentration (MIC) defined as the last concentration without microbial growth was determined. The microdilution susceptibility assay was performed with the nanoconjugates and nanoparticles for n=2-3 experiments each.

Minimum Inhibitory Concentration (MIC) with Others Bacteria Strains

Minimum inhibitory concentration (MIC) for tobramycin (Tob), gentamycin, colistin and the corresponding nanoconjugates; and antimicrobial susceptibility test assays were performed according to the Clinical and Laboratory Standards Institute (CLSI) guidelines M07-A10 Methods for Dilution Antimicrobial Susceptibility Tests for Bacteria That Grow Aerobically, 10^{th} Edition. All test and quality control strains were recovered from frozen storage (−80° C.) and cultured at 35±2° C. for 18-24 h.

All test articles were dissolved in sterile ultra-pure water at a concentration of 2.56 mg/mL. Tobramycin (Cayman Chemical; #14596) was assumed to have a potency of 1000 μg/mg; all other test articles were adjusted according to the amount of tobramycin per total amount of compound, so that the total concentration of tobramycin equaled 2.56 mg/mL. In sterile 96-well flat-bottomed plates (Corning; #3370), test articles were further two-fold serially diluted in cation-adjusted Mueller-Hinton broth (CA-MHB; Oxoid; CM0405) to provide a test concentration range of 0.25-128 μg/mL, or 0.125-64 μg/mL for quality control strains against tobramycin. 100 and 200 μL of drug-free medium was added to positive and negative control wells, respectively. Suspensions equivalent to a McFarland 0.5 standard were prepared for each test strain in 5 mL sterile phosphate-buffered saline (PBS) and further diluted 1:150 in CA-MHB to provide an inoculum of 2-8×10⁵ CFU/mL. 100 μL of each inoculum was dispensed into all wells—except negative control wells (100 μL sterile water added)—to provide an inoculum of ˜5×10⁵ CFU/well and a final volume of 200 μL per well. Assay plates were incubated according to CLSI M07-A10 guidelines of 35±2° C. for 16-20 h in air. After incubation, plates were read by spectrophotometer at 600 nm, and the minimum inhibitory concentration (MIC) was determined as the lowest concentration of drug giving 100% inhibition of visual growth compared to that of the drug-free control.

TKK experiments performed for a culture of P. aeruginosa ATCC 27853 prepared by inoculating 20 mL MHB with a single, well isolated colony. Stock solutions of 100 times the required final concentration were prepared for two replicate experiments and stored at −20° C. Before use, drug stocks were thawed at 42° C. for 5-10 mins.

Assays were performed in sterile Universal glass vials containing a final volume of 10 mL cation-adjusted Mueller Hinton broth (caMHB). A starter culture of P. aeruginosa ATCC 27853 was prepared by inoculating 20 mL MHB with a single, well isolated colony. Following overnight (16 h) incubation at 37° C. and 180 rpm, the culture was subjected to centrifugation at 4000 g, the supernatant removed and the bacterial pellet re-suspended in an equal volume sterile phosphate-buffered saline (PBS). The resulting suspension was then adjusted to that equivalent to a McFarland 0.5 standard (˜1×10⁸ CFU/mL) and back-diluted 1:200 into 10 mL fresh caMHB broth containing 0.5, 1, 2, 5, or 10× the MIC of free antibiotics and their corresponding nanoconjugates of the present invention. This provided a starting inoculum of ˜5×10⁵ CFU/mL, which was confirmed by a total viable count performed immediately at T=0 on the drug-free untreated control broth. All broths were incubated at 37° C. and at 180 rpm, and total viable counts determined at 1, 2, 4, 6, 8, and 24 h post-inoculation. Samples were withdrawn at each time point, washed twice in sterile PBS following centrifugation at room temperature and 16,000 g for one minute, serially 10-fold diluted in PBS and plated onto Luria-Bertani (LB) agar (10 g/L sodium chloride, 10 g/L tryptone, 5 g/L yeast extract, 15 g/L bacteriological agar). Plates were then incubated in ambient air at 37° C. for up to 24 h, following which the number of viable P. aeruginosa (CFU/mL) was determined.

Experiments were performed in duplicate on separate days (n=2). The limit of detection (LoD) for these assays was 50 CFU/mL. Bactericidal activity refers to ≥3 log₁₀ reduction in viability relative to the starting inoculum after 24 h exposure.

### A. Dextran Methacrylate (DXT-MA) Based Nanoconjugates

### A.1. Single-Chain Dextran Methacrylate Based Nanoparticles (DXT-SCPN)

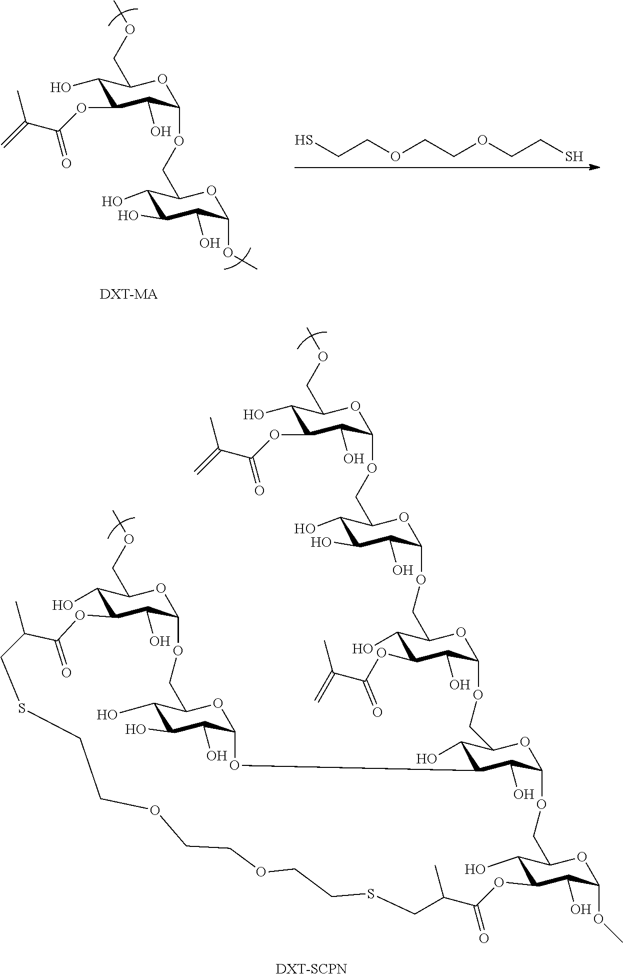

### Example 1. DXT-SCPN1 HAVING 10% OF INTRAMOLECULAR CROSSLINKING BASED ON THE AMOUNT OF Methacrylate Group Present in the DXT-MA from DXT40-MA

0.37 mL (0.05 mmol) of a 0.15 M solution of the cross-linker 3,6-dioxa-1,8-octane-dithiol (DODT) (98 NL of DODT in 4 mL of MeOH/PBS, 1:1, v/v, pH=9.5) was added dropwise using a syringe pump (0.04 mL/h) over a 0.02 M solution of dextran methacrylate (DXT40-MA; having a substitution degree (DS) of 52%) (500 mg, 1.065 mmol, 50 mL PBS, pH=9.5) during 8 h at room temperature and under constant stirring.

After addition, the reaction was maintained stirred at room temperature for 12 h. Further characterization studies were carried out after purification of the reaction mixture using a stirred cell purification system with distilled water (3×300 mL). Finally the resulting aqueous solution was freeze-dried to obtain DXT-SCPN1 as a white solid. Yield >90%. ¹H NMR (500 MHz, D₂O) δ ppm: 6.34-6.12 (m, 1H, methacrylic-CH), 5.94-5.70 (m, 1H, methacrylic-CH), 5.55-4.85 (5.5H, including H-1 and H-2/3 MA-substituted), 4.34-3.28 (28H, m, rest of Glc and 2×CH₂O of crosslinker), 3.06-2.53 (5H, m, CH(CH₃)CH₂S, CH₂S of cross-linker), 1.98 (s, 3H, methacrylic-CH₃), 1.29 (s, 3H, cross-linker-CH₃). Dh (DLS)=16±10 nm; PDI (size) 0.4.

### Example 2. DXT-SCPN2 Having 50% of Intramolecular Crosslinking Based on the Amount of Methacrylate Group Present in the DXT-MA from DXT40-MA

3.8 mL (0.55 mmol) of a 0.15 M solution of cross-linker 3,6-dioxa-1,8-octane-dithiol (DODT) (98 μL of DODT in 4 mL of MeOH/PBS, 1:1, v/v, pH=9.5) was added dropwise using a syringe pump (0.5 mL/h) over a 0.02 M solution of dextran methacrylate (DXT40-MA; DS=50%) (1 g, 2.21 mmol, 100 mL PBS, pH=9.5) during 8 h at room temperature and under constant stirring. After addition, the reaction was maintained stirred at room temperature for 12 h. Further characterization studies were carried out after purification of the reaction mixture using a stir cell purification system with distilled water (3×300 mL). Finally, the resulting aqueous solution was freeze-dried to obtain DXT-SCPN2 as a white solid. Yield >90%. ¹H NMR (500 MHz, D₂O) δ ppm: 6.34-6.12 (m, 1H, methacrylic-CH), 5.94-5.70 (m, 1H, methacrylic-CH), 5.55-4.85 (5.5H, including H-1 and H-2/3 MA-substituted), 4.34-3.28 (28H, m, rest of Glc and 2×CH₂O of crosslinker), 3.06-2.53 (5H, m, CH(CH₃)CH₂S, CH₂S of cross-linker), 1.98 (s, 3H, methacrylic-CH₃), 1.29 (s, 3H, cross-linker-CH₃).Dh (DLS)=13±11 nm; PDI (size) 0.3.

### Example 3: DXT-SCPN3 Having 10% of Intramolecular Crosslinking Based on the Amount of Methacrylate Group Present in the DXT-MA from DXT20-MA

0.08 mL (0.007 mmol) of a 0.08 M solution of the cross-linker 3,6-dioxa-1,8-octane-dithiol (DODT, 48 NL of DODT in 4 mL of MeOH/PBS, 1:1, v/v, pH=9.5) was added dropwise using a syringe pump (0.01 mL/h) over a 0.015 M solution of dextran methacrylate (DXT20-MA; having a substitution degree (DS) of 28%) (100 mg, 0.15 mmol, 10 mL PBS, pH=9.5) during 8 h at room temperature and under constant stirring.

After addition, the reaction was maintained stirred at room temperature for 16 h. Further characterization studies were carried out after purification of the reaction mixture by dialysis using regenerated cellulose membranes with a molecular weight cut off (MWCO) of 3.5 kDa. Water is replaced until water conductivity reaches values lower than 1 μS·cm⁻¹. Finally the resulting aqueous solution was freeze-dried to obtain DXT-SCPN3 as a white solid. Yield >90%. ¹H NMR (500 MHz, D₂O) δ ppm: 6.34-6.12 (m, 1H, methacrylic-CH), 5.94-5.70 (m, 1H, methacrylic-CH), 5.55-4.85 (5.5H, including H-1 and H-2/3 MA-substituted), 4.34-3.28 (28H, m, rest of Glc and 2×CH₂O of crosslinker), 3.06-2.53 (5H, m, CH(CH₃)CH₂S, CH₂S of cross-linker), 1.98 (s, 3H, methacrylic-CH₃), 1.29 (s, 3H, cross-linker-CH₃). Dh (DLS)=10±5 nm; PDI (size) 0.3.

### Example 4: DXT-SCPN4 Having 60% of Intramolecular Crosslinking Based on the Amount of Methacrylate Group Present in the DXT-MA from DXT70-MA

0.83 mL (0.125 mmol) of a 0.15 M solution of the cross-linker 3,6-dioxa-1,8-octane-dithiol (DODT, 49 μL of DODT in 2 mL of MeOH/PBS, 1:1, v/v, pH=9.5) was added dropwise using a syringe pump (0.1 mL/h) over a 0.015 M solution of dextran methacrylate (DXT70-MA; having a substitution degree (DS) of 60%) (184 mg, 0.5 mmol, 18.4 mL PBS, pH=9.5) during 8 h at room temperature and under constant stirring.

After addition, the reaction was maintained stirred at room temperature for 16 h. Further characterization studies were carried out after purification of the reaction mixture by dialysis using regenerated cellulose membranes with a molecular weight cut off (MWCO) of 3.5 kDa. Water is replaced until water conductivity reaches values lower than 1 μS·cm⁻¹. Finally the resulting aqueous solution was freeze-dried to obtain DXT-SCPN4 as a white solid. Yield >90%. ¹H NMR (500 MHz, D₂O) δ ppm: 6.47-5.96 (m, 1H, methacrylic-CH), 5.90-5.53 (m, 1H, methacrylic-CH), 5.49-4.77 (5.5H, including H-1 and H-2/3 MA-substituted), 4.47-3.06 (28H, m, rest of Glc and 2×CH2O of crosslinker), 2.99-2.36 (5H, m, CH(CH3)CH2S, CH2S of cross-linker), 1.89 (s, 3H, methacrylic-CH3), 1.20 (s, 3H, cross-linker-CH3). Dh (DLS)=18±9 nm; PDI (size) 0.17.

### A.2. Functionalization of DXT-SCPN with 3-Mercaptopropionic Acid (DXT-SCPN-F)

### Example 5. DXT-SCPN1-F1 (from DXT-SCPN1 of Example 1)

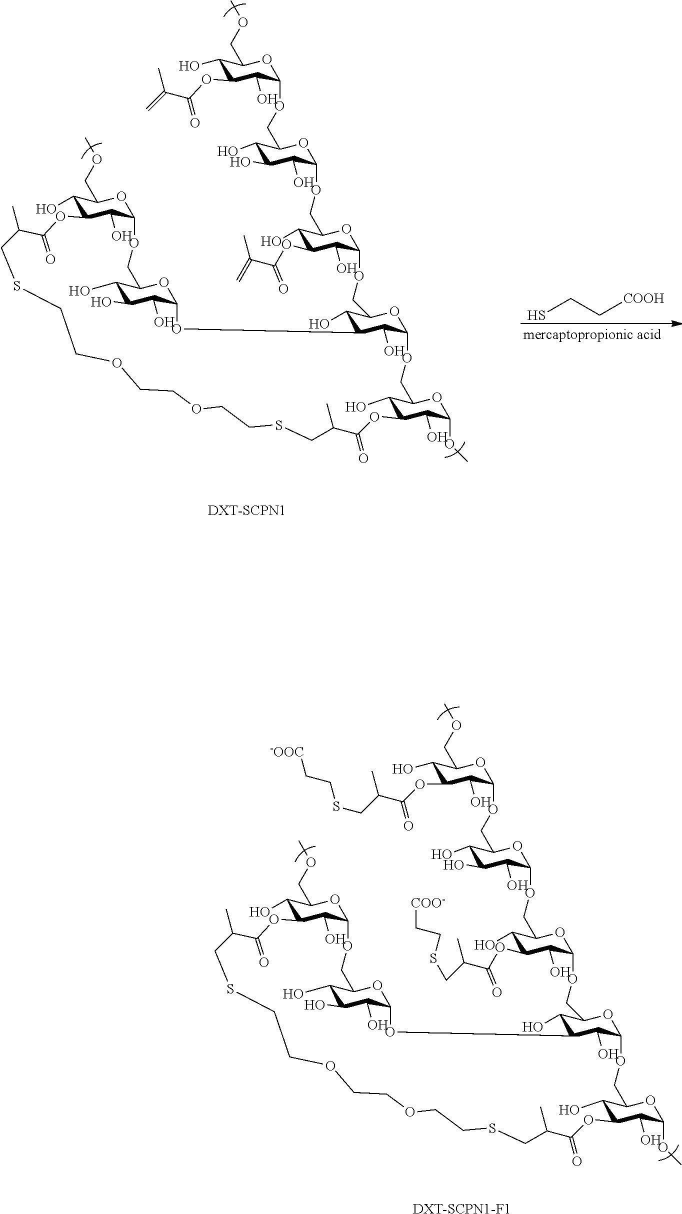

The synthesis of the functionalized DXT-SCPN1-F1 was achieved by adding slowly 1.25 mL of a freshly prepared aqueous solution of 3-mercaptopropionic acid (615 NL, 1885 NL H₂O, pH=9.5) to the reaction flask in the DXT-SCPN1 obtained in Example 1. The reaction was stirred for 5 h and the excess acid was removed by dialysis against distilled water (MWCO 3.5 kDa) until reaching deionized water conductivity values <1 μS·cm⁻¹ (5 days, refreshing with 4 L of deionized water twice per day). The resulting aqueous solution was freeze-dried to obtain functionalized nanoparticles DXT-SCPN1-F1 as a white solid. Yield >90%. GPC-SEC characterization for the DXT-SCPN1-F1 was carried out and the DXT-SCPN1-F1 was compared with DXT-F1 (DXT-MA functionalized with mercaptopropionic acid) used as starting material and DXT-SCPN1 of Example 1 to confirm the increase of the retention time due to the reduction of the hydrodynamic radius as evidence of the polymer compaction into SCPN: DXT-SCPN1-F1, Mw (GPC)=77 KDa, Mw/Mn=1.4 Dh (DLS)=22±7 nm; PDI (size) 0.4. Z-potential=−26±3 mV.

### Example 6. DXT-SCPN2-F2 (from DXT-SCPN2 of Example 2)

The synthesis of the functionalized DXT-SCPN2-F2 was achieved by adding slowly 2.5 mL of a freshly prepared aqueous solution of 3-mercaptopropionic acid (615 μL, 1885 μL H₂O, pH=9.5) to the reaction flask in the DXT-SCPN2 obtained in Example 2. The reaction was stirred for 5 h and the excess acid was removed by dialysis against distilled water (MWCO 3.5 kDa) until reaching deionized water conductivity values <1 μS·cm⁻¹ (5 days, refreshing with 4 L of deionized water twice per day). The resulting aqueous solution was freeze-dried to obtain functionalized nanoparticles DXT-SCPN2-F2 as a white solid. Yield >90%. GPC-SEC characterization for the DXT-SCPN2-F2 was carried out and the DXT-SCPN2-F2 was compared with DXT-MA used as starting material and DXT-SCPN2 of Example 2 to confirm the increase of the retention time due to the reduction of the hydrodynamic radius as evidence of the polymer compaction into SCPN2: DXT-SCPN2-F2, Mw (GPC)=70 KDa, Mw/Mn=1.4. Dh (DLS)=15±4 nm; PDI (size) 0.2. Z-potential=−22±6 mV

### Example 7. DXT-SCPN3-F3 (from DXT20-SCPN3 of Example 3)

The synthesis of the functionalized DXT-SCPN3-F3 was achieved by adding slowly 0.2 mL of a freshly prepared aqueous solution of 3-mercaptopropionic acid (615 NL, 2500 NL H₂O, pH=9.5) to the reaction flask in the DXT-SCPN3 obtained in Example 3. The reaction was stirred for 5 h and the excess acid was removed by dialysis against distilled water (MWCO 3.5 kDa) until reaching deionized water conductivity values <1 μS·cm⁻¹ (5 days, refreshing with 4 L of deionized water twice per day). The resulting aqueous solution was freeze-dried to obtain functionalized nanoparticles DXT-SCPN3-F3 as a white solid. Yield >90%. ¹H NMR (500 MHz, D₂O) δ ppm, 5.34-4.99 (9H, including H-1 and H-2/3 MA-substituted), 4.00-3.53 (53H, m, rest of Glc and 2×CH₂O of crosslinker), 2.92-2.53 (4.8H, m, CH(CH₃)CH₂S, CH₂S of cross-linker and MPA), 1.32 (s, 2.5H, MPA and cross-linker-CH₃). Dh (DLS)=10±6 nm; PDI (size) 0.3. Zeta potential: −16±6 mV.

### Example 8. DXT-SCPN4-F4 (from DXT70-SCPN4 of Example 4)

The synthesis of the functionalized DXT (70 kDa)-SCPN4-F4 was achieved by adding slowly 886 NL of a freshly prepared aqueous solution of 3-mercaptopropionic acid (615 NL, 2500 NL H₂O, pH=9.5) to the reaction flask in the DXT-SCPN4 obtained in Example 4. The reaction was stirred for 5 h and the excess acid was removed by dialysis against distilled water (MWCO 3.5 kDa) until reaching deionized water conductivity values <1 S·cm⁻¹ (5 days, refreshing with 4 L of deionized water twice per day). The resulting aqueous solution was freeze-dried to obtain functionalized nanoparticles DXT-SCPN4-F4 as a white solid. Yield >90%. ¹H NMR (500 MHz, D₂O) δ ppm, 5.46-4.79 (5.5H, including H-1 and H-2/3 MA-substituted), 4.22-3.24 (28H, m, rest of Glc and 2×CH₂O of crosslinker), 3.02-2.66 (8H, m, 2×CH(CH₃)CH₂S, CH₂S of cross-linker and MPA), 2.66-2.39 (2H, m, CH₂COOH of MPA), 1.22 (s, 3H, cross-linker-CH₃). Zeta Potential=−22±4 mV.

### A.3. Nanoconjugates of DXT-SCPN

### Example 9. Nanoconjugate of Tobramycin with Non-Covalent Bond (Ionic Bond)-Tob-DXT-SCPN1-Ionic (from DXT-SCPN1-F1 and Tobramycin)

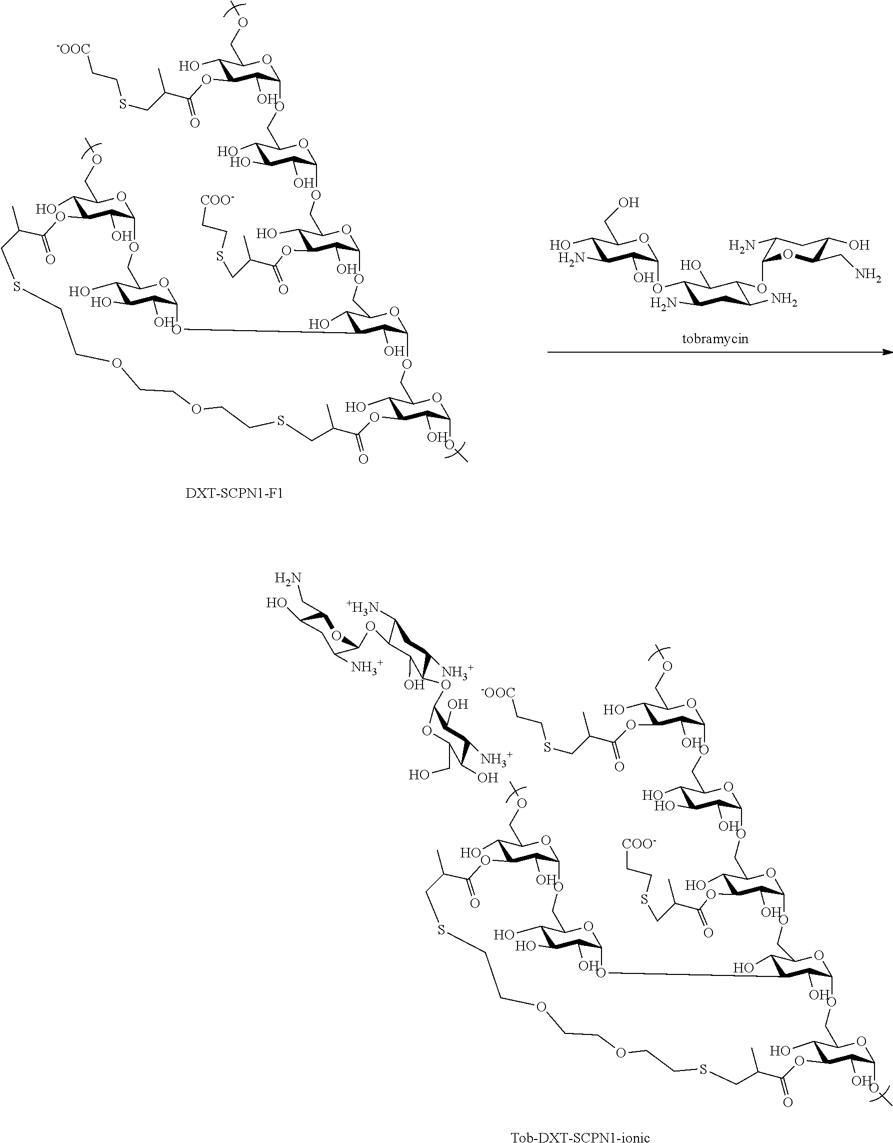

The preparation of Tob-DXT-SCPN1 was performed by dispersing 10 mg of DXT-SCPN1-F1 of Example 5 in 1.2 mL of milliQ water for 1 hour. Then, Tobramycin was added until reaching neutral Z-potential (Z-potential=0±5 mV) at pH=7. The mixture was stirred for 1 hour and then freeze-dried (−42° C., 0.120 mBar, 24 h) to obtain the nanoconjugate Tob-DXT-SCPN 1-ionic as a white powder. ¹H NMR (500 MHz, D₂O) δ ppm: 5.66-5.65 (d, 1H), 5.18-4.99 (m, 11.8H), 4.00-3.42 (m, 78H), 3.22-3.18 (m, 2H), 2.92-2.76 (m, 17.3H), 2.50-2.47 (m, 6.2H), 2.31-2.27 (m, 2.2H), 1.67-1.59 (m, 1.1H), 1.29-1.28 (m, 9.9H), 1.17-1.14 (m, 0.7H). Dh (DLS)=13±6 nm; PDI (size) 0.13. Zeta potential: −3±3 mV.

Tobramycin Loading=40% by weight of tobramycin in relation to the total weight of the nanoconjugate.

### Example 10. Nanoconjugate of Tobramycin with Non-Covalent Bond (Ionic Bond)-Tob-DXT-SCPN2-Ionic (from DXT-SCPN2-F2 and Tobramycin)

The preparation of Tob-DXT-SCPN2 was performed by dispersing 10 mg of DXT-SCPN2-F2 of Example 6 in 1.2 mL of milliQ water for 1 hour. Then Tobramycin was added until reaching neutral Z-potential (Z-potential=0±5 mV) at pH 7. The mixture was stirred for 1 hour and then freeze-dried (−42° C., 0.120 mBar, 24 h) to obtain the nanoconjugate Tob-DXT-SCPN 2-ionic as a white powder.

Tobramycin Loading=24 wt % by weight of tobramycin in relation to the total weight of the nanoconjugate.

### Example 11. Nanoconjugate of Colistin with Non-Covalent Bond (Ionic Bond)-Col-DXT-SCPN4-Ionic (from DXT-SCPN1-F1 and Colistin)

The preparation of Col-DXT-SCPN1 was performed by dispersing 15 mg of DXT-SCPN1-F1 of Example 5 in 1.2 mL of milliQ water for 1 hour. Then, Colistin sulfate was added and the pH was adjusted to pH 7. The mixture was stirred for 1 hour and then freeze-dried (−42° C., 0.120 mBar, 24 h) to obtain the nanoconjugate Col-DXT-SCPN4-ionic as a white powder.

Colistin Loading=25 wt % by weight of colistin in relation to the total weight of the nanoconjugate. Z potential: −8±3 mV.

### Example 12. Nanoconjugate of Gentamycin with Non-Covalent Bond (Ionic Bond)-Gen-DXT-SCPN5-Ionic (from DXT-SCPN1-F1 and Gentamicin)

The preparation of Gen-DXT-SCPN5 was performed by dispersing 15 mg of DXT-SCPN1-F1 of Example 5 in 1.2 mL of milliQ water for 1 hour. Then, gentamycin sulfate was added and the pH was adjusted to pH 7. The mixture was stirred for 1 hour and then freeze-dried (−42° C., 0.120 mBar, 24 h) to obtain the nanoconjugate Gen-DXT-SCPN5-ionic as a white powder.

Gentamycin Loading=25 wt % of Gentamycin in relation to the total weight of the nanoconjugate Z potential: −8±4 mV.

### Example 13. Nanoconjugate of Tobramycin with Hydrolysable Covalent Bond-Tob-DXT-SCPN1-Cov (from DXT-SCPN1 and Tobramycin)

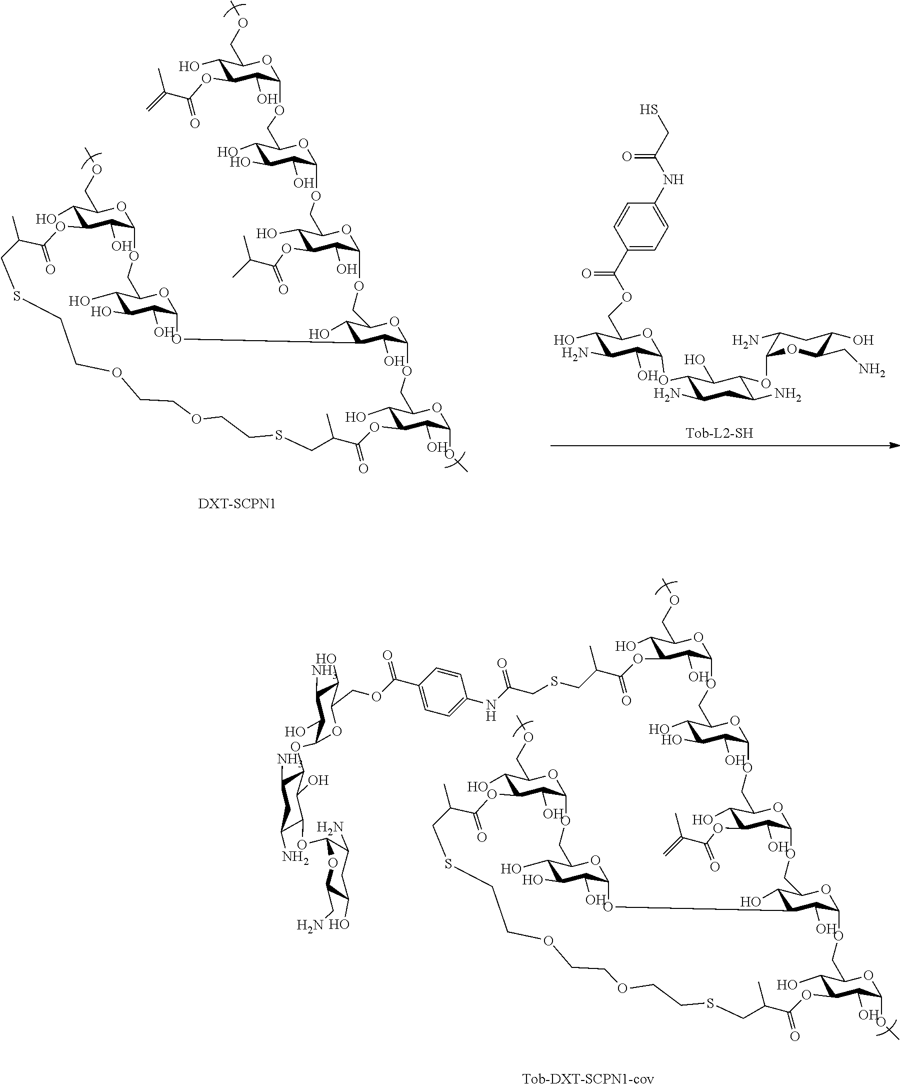

The preparation of Tob-DXT-SCPN1-cov was performed by dispersing 10 mg of DXT-SCPN1 of Example 1 in 1 mL of PBS at pH 8 for 1 hour. Meanwhile, 20 mg of Tob-L₂-SH (TFA salt) was dissolved in PBS pH8 and the pH was adjusted to 8 by dropwise addition of 0.1N NaOH aqueous solution. Both solutions were mixed and stirred for 16 hours at room temperature and under nitrogen atmosphere. Then, the crude was purified by exclusion chromatography (Sephadex G-25 PD-10 columns) and then freeze-dried (−42° C., 0.120 mBar, 24 h) to obtain the Tob-DXT-SCPN3 as a white to yellowish powder. ¹H NMR (500 MHz, D₂O) δ ppm: 7.89-7.41 (m, 4H, Harom), 6.13-5.92 (m, 0.5H), 5.66-5.53 (m, 0.5H), 5.22-4.75 (m, 8.4H), 4.30-4.00 (m, 3H), 3.81-3.20 (m, 46.5H), 3.10-2.44 (9.5H), 1.99-1.85 (m, 1.8H), 1.83-1.66 (m, 1.9H), 1.63-1.48 (m, 0.8H), 1.41-1.21 (m, 1.1H), 1.18-0.84 (m, 4.4H). Dh (DLS)=14±5 nm; PDI (size) 0.3. Zeta potential: +7±3 mV.

Tobramycin Loading=29 wt % by weight of tobramycin in relation to the total weight of the nanoconjugate.

Conjugation efficiency=72%. MA group that have reacted=62%.

### A.4. Pharmacokinetic Evaluation and Activity Tests of the Nanoconjugates of DXT-SCPN

### Example 14. Release Profile Test of Tob-DXT-SCPN

Tob-DXT-SCPN1-Ionic

Samples:
Tobramycin
Tob-DXT-SCPN1-ionic

Method

Tobramycin release from Tob-DXT-SCPN1-ionic was evaluated in PBS at pH 7.4. 1 mL dialysis tubes (Float-a-lyzer®) were used whose membrane is based on MWCO 8-10 kDa cellulose ester. These tubes were filled with at least 5 mg of neat tobramycin in 1 mL of PBS and placed in 50 mL falcons. The studies were performed in a chamber at 37° C. and stirring at 250 rpm. Samples were taken at different times and analyzed by LC-MS.

Results

The release profile of Tobramycin from Tob-DXT-SCPN1-ionic in PBS at pH 7.4 is reported in the Table below. The term Tobramycin released (%) expresses the percentage of tobramycin free or liberated from the Tob-DXT-SCPN1-ionic of the present invention that release the dialysis tubes in each tested time.

| | Tobramycin | | Tob-DXT-SCPN1-ionic | |
|---|---|---|---|---|
| Time | Tobramycin | | tobramycin | |
| (hour) | released (%) | SD | released (%) | SD |
| 0 | 0.30 | 0.43 | 0 | 0 |
| 1 | 2.91 | 2.09 | 1.90 | 1.67 |
| 2 | 7.57 | 6.00 | 6.30 | 1.08 |
| 4 | 19.03 | 8.07 | 9.27 | 10.12 |
| 8 | 28.72 | 15.32 | 28.95 | 3.86 |
| 24 | 58.41 | 18.66 | 63.43 | 7.76 |
| 48 | 78.88 | 6.10 | 84.27 | 16.13 |
| 72 | 87.20 | 0.28 | 86.83 | 20.65 |

SD stands for standard deviation

The results of the Table show that the antibiotic tobramycin was released from the nanoconjugate of the present invention in such a way that allows having in 24 h about 60% of release and in 48 h about 80% of release as the tobramycin free. It means that the conjugates of the present invention allows having a bioavailability comparable to the tobramycin free.

Tob-DXT-SCPN1-Cov

Samples:
Tobramycin
Tob-DXT-SCPN1-cov

Method

Tobramycin release from Tob-DXT-SCPN1-cov was evaluated in PBS at pH 7.4. 1 mL dialysis tubes (Float-a-lyzer®) were used whose membrane is based on MWCO 8-10 kDa cellulose ester. These tubes were filled with at least 5 mg of neat tobramycin in 1 mL of PBS and placed in 50 mL falcons. The studies were performed in a chamber at 37° C. and stirring at 250 rpm. Samples were taken at different times and analyzed by LC-MS.

Results

The release profile of Tobramycin from Tob-DXT-SCPN1-cov in PBS at pH 7.4 is reported in the Table below:

| | Tob-L2-S- | free | Tobramycin |
|---|---|---|---|
| Time | CH₂-CH(CH₃)- | Tobramycin | total |
| (hour) | COOH released (%) | released (%) | released (%) |
| 0 | 0 | 0 | 0 |
| 1 | 0 | 0 | 0 |
| 2 | 0 | 0.12 | 0 |
| 4 | 0.48 | 0.42 | 1 |
| 8 | 0.99 | 1.04 | 2 |
| 24 | 3.85 | 5.08 | 9 |
| 48 | 9.77 | 16.90 | 27 |
| 72 | 8.04 | 15.19 | 23 |

Two different species are released from the conjugate of the present invention (Tob-DXT-SCPN1-cov). These species are free tobramycin and tobramycin-linker (Tob-L2-S—CH₂—CH(CH₃)—COOH). As it is shown in the results of the Table above, free tobramycin is the majority compound released. Furthermore, tobramycin-linker (Tob-L2-S—CH₂—CH(CH₃)—COOH) will eventually decompose into free tobramycin and the linker (H-L2-S—CH₂—CH(CH₃)—COOH). The results of the Table also show that the antibiotic tobramycin was released from the nanoconjugate of the present invention in such a way that allows having in 24 h about 5% of release and in 48 h about 17% of release.

Finally, it was also confirmed that no changes in functionalities or chemical groups of tobramycin free occurred when it is linked to the single-chain dextran methacrylate or acrylate based nanoparticles disclosed in the present invention to give the corresponding nanoconjugates Tob-DXT-SCPN1-cov. Then, the active ingredient (tobramycin) in the nanoconjugates of the present invention is chemically identical to the free active ingredient.

### Example 15. Antibiotic Susceptibility Test of the DXT-SCPNs of the Present Invention

MIC of the free hydrophilic tobramycin and its nanoconjugates of the present invention were performed. The MIC values obtained for Tobramycin free, Tob-DXT-SCPN 1-ionic and DXT-SCPN1 of the present invention are summarised in the Table below:

| | | Test Article MIC (μg/mL) | | |
|---|---|---|---|---|
| | | | Tob-DXT- | DXT- |
| | | Free | SCPN1- | SCPN- |
| Strain | Replicate | Tobramycin | ionic | F1 |
| E. coli | 1 | 2^{a} | 4 | >128 |
| ATCC 25922 | 2 | 2^{a} | 4 | >128 |
| E. coli | 1 | >128 | >128 | >128 |
| ATCC | 2 | >128 | >128 | >128 |
| BAA-2469 | | | | |
| P. aeruginosa | 1 | 1^{b} | 2 | >128 |
| ATCC 27853 | 2 | 1^{b} | 1 | >128 |
| P. aeruginosa | 1 | 128 | >128 | >128 |
| ATCC 13437 | 2 | 64 | >128 | >128 |
| A. baumannii | 1 | 0.5 | 1 | >128 |
| ATCC | 2 | 0.5 | 1 | >128 |
| BAA-747 | | | | |
| A. baumannii | 1 | 4 | 8 | >128 |
| NCTC 12156 | 2 | 4 | 16 | >128 |
| K. pneumoniae | 1 | 0.5 | 1 | >128 |
| ATCC 43816 | 2 | 0.5 | 1 | >128 |
| K. pneumoniae | 1 | >128 | >128 | >128 |
| ATCC | 2 | >128 | >128 | >128 |
| BAA-2146 | | | | |
| E. aerogenes | 1 | 4 | 8 | >128 |
| KPC 108 | 2 | 4 | 8 | >128 |
| E. cloacae | 1 | 32 | 64 | >128 |
| complex | 2 | 32 | 128 | >128 |
| KPC 114 | | | | |

^{a}One doubling diltution above CLSI M100 S27 QC range (0.25-1 μg/mL)
^{b}Within CLSI M100 S27 QC range (0.25-1 μg/mL)

The results of the Table above shows that DXT-SCPN alone did not show any antimicrobial activity. On the other hand, the tobramycin attached ionically to DXT-SCPN1 (i.e Tob-DXT-SCPN 1-ionic) showed relatively good performances against the right bacteria strain affected by tobramycin. MIC of Tob-DXT-SCPN1-ionic was found to double compared to free antibiotic but this fairly good results showing that the use of the single-chain dextran methacrylate or acrylate based nanoparticle as defined in the present invention did not significantly affect the activity of tobramycin.

Further, the MIC values obtained for Tobramycin free, Tob-DXT-SCPN1-ionic, DXT-SCPN2-ionic as well as for colistin and gemtamycin free and their corresponding nanoconjugates of the present invention Co-DXT-SCPN4-ionic and Gen-DXT-SCPN5-ionic of the present invention are summarised in the Table below:

| | S. aureus | | P. aeruginosa | |
|---|---|---|---|---|
| | | Nano- | | Nano- |
| | Antibiotic | conjugate | Antibiotic | conjugate |
| | conc. | conc. | conc. | conc. |
| MIC 50 | (μg/ml) | (μg/ml) | (μg/ml) | (μg/ml) |
| Free Tobramycin (Tob) | 0.5-1 | — | 0.5-1 | — |
| Tob-DXT-SCPN1-ionic | 0.5-1 | 2-4 | 0.5-1 | 2-4 |
| (Example 9) | | | | |
| Tob-DXT-SCPN2-ionic | 0.5-1 | 2.08-4.17 | 0.5-1 | 2.08-4.17 |
| (Example 10) | | | | |
| Free Colistin sulfate (Col) | NA | — | 1-5 | — |
| Col-DXT-SCPN4-ionic | NA | — | 1-5 | 4-20 |
| (Example 11) | | | | |
| Free Gentamycin sulfate | 1-5 | — | 1-5 | — |
| (Gen) | | | | |
| Gen-DXT-SCPN5-ionic | 1-5 | 4-20 | 1-5 | 4-20 |
| (Example 12) | | | | |
| DXT-SCPN1 (Example 1) | NA | — | NA | — |
| DXT-SCPN2 (Example 2) | NA | — | NA | — |

NA stands for not available.

TKK experiments for Tob-DXT-SCPN1 and performed for a culture of P. aeruginosa ATCC 27853 prepared by inoculating 20 mL MHB with a single, well isolated colony. Stock solutions of 100 times the required final concentration were prepared for two replicate experiments and stored at −20° C. Before use, drug stocks were thawed at 42° C. for 5-10 mins.

It is interesting to point that even at a concentration corresponding to 0.5 of the MIC previously determined; Tob-DXT-SCPN1 of the present invention was able to kill a large quantity of bacteria within 1 hrs while after 2 hours similar bacteria level were achieved compared to concentration corresponding to 1 and 10 time the MIC concentration. It has to be noted that after 24 hrs a slight increase of bacteria concentration is also observed, indicating that at 24 hrs timescale, 0.5 MIC concentration might not be enough. On the other hand, it is interesting to mention that even at the exact MIC concentration no further increase in bacteria population could be observed. Despite the need to longer time scale experiments it is encouraging to see that the minimum amount of Tob-DXT-SCPN no hydrolysable covalent is sufficient during 24 hrs.

### Example 16. Testing Against Biofilm of Pseudomonas aeruginosa PAO1

Samples:
Tobramycin
Tob-DXT-SCPN1-ionic

Method:

Continuous biofilm of Pseudomonas Aeruginosa PAO1 gfp in LB medium was grown for 3 days. Biofilm was treated for 12 h with 200 μL of samples (tobramycin concentration 2 μg/mL). Images were obtained using a 20×/0.80 air objective with Zeiss LSM 800 Confocal laser scanning microscope. Simulated fluorescence projections and orthogonoal views sections were processed using ImageJ software. Data were analysed with COMSTAT2 software.

Results:

| Samples | % Biomass (μm³/μm²) | p-value |
|---|---|---|
| Tobramycin | 68.1% | <0.0001 vs control |
| Tob-DXT- | 60.3% | <0.0001 vs control |
| SCPN1-ionic | | <0.05 vs Tobramycin |

Reduction of the biomass is observed after treatment with both tobramycin and Tob-DXT-SCPN1-ionic. Unexpectedly, the effect on the production of biomass for the biofilm is greater for Tob-DXT-SCPN1-ionic (60.3% biomass vs 68.1% biomass), which means that the nanoconjugate impede biofilm to grow correctly.

### B. Dextran Acrylate (DXT-A) Based Nanoconjugates

### B.1 Dextran Acrylate (DXT-A)

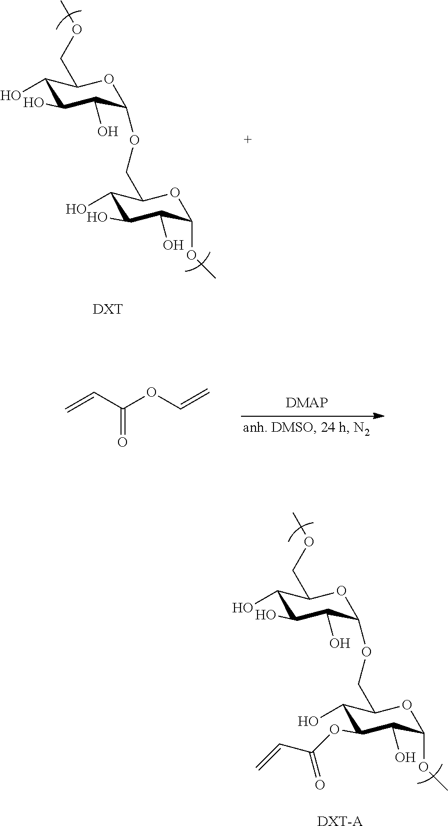

### Example 17. DXT-A

DXT40 (200 mg, 1.2 mmol Glc) was dissolved into 5 mL of anhydrous DMSO under N₂ atmosphere. Then, 4-(dimethylamino)pyridine (DMAP) (7 mg, 0.06 mmol) was poured into the flask. After complete dissolution, vinyl acrylate (120 mL, 1.2 mmol) was added dropwise and the reaction mixture was stirred overnight (16 h) at 50° C. under N₂ atmosphere. DMAP was quenched by addition of HCl 0.1M (0.06 mmol) and the resulting polymer was purified using a stirred cell purification system with distilled water (5×300 mL). The resulting aqueous solution was freeze-dried to obtain DXT-A as a white solid. Yield >90%. DS=47%. ¹H NMR (500 MHz, D₂O) δ ppm: 6.45-5.96 (m, 3H, acrylic-CH), 5.17-4.82 (2.7H, including H-1 and H-2/3 A-substituted Glc protons), 3.90-3.45 (11.6H, m, rest of Glc and 2×CH₂O of crosslinker).

### B.2. Single-Chain Dextran Acrylate Based Nanoparticles (DXT-A-SCPN)

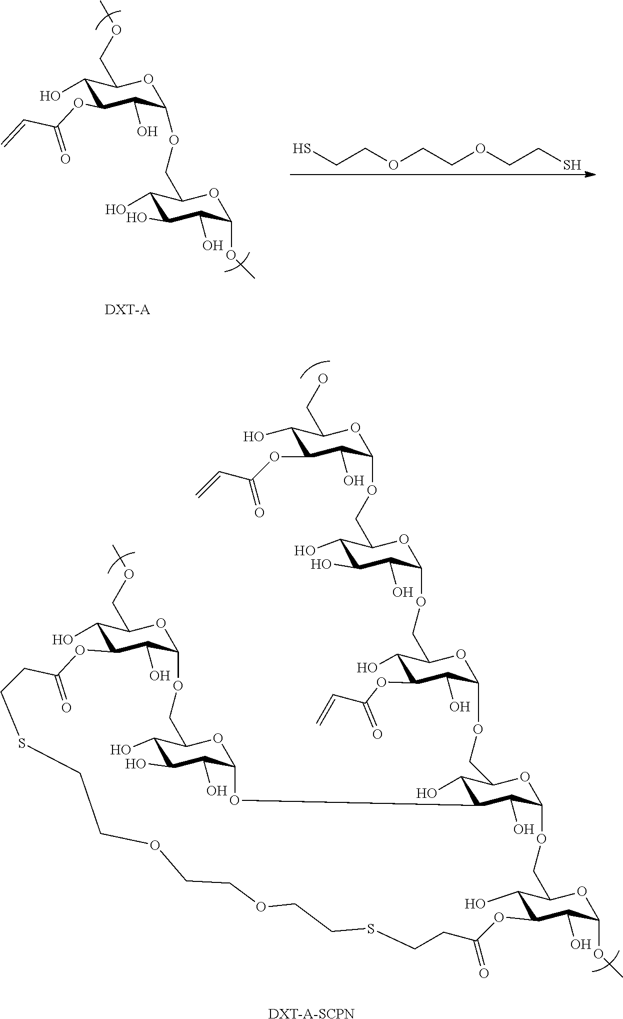

### Example 18. DXT-A-SCPN1 from DXT40-A

0.89 mL (0.013 mmol) of a 0.015 M solution of the cross-linker 3,6-dioxa-1,8-octane-dithiol (DODT) (4.9 NL of DODT in 2 mL of MeOH/PBS, 1:1, v/v, pH=8) was added dropwise using a syringe pump (0.11 mL/hour) over a 0.02 M solution of dextran acrylate (DXT40-A; having a substitution degree (DS) of 50%) (50 mg, 1.065 mmol, 5 mL PBS, pH=8) during 8 h at room temperature and under constant stirring.

Further characterization studies were carried out after purification of the reaction mixture using a stirred cell purification system with distilled water (3×300 mL). The resulting aqueous solution was freeze-dried to obtain DXT-A-SCPN1 as a white solid. Yield >90%. ¹H NMR (500 MHz, D₂O) δ ppm: 6.44-5.96 (m, 3H, acrylic-CH), 5.16-4.90 (4.3H, including H-1 and H-2/3 A-substituted Glc protons), 3.89-3.43 (20.7H, m, rest of Glc and 2×CH₂O of crosslinker), 2.81-2.73 (3.3H, m, CH(CH₃)CH₂S, CH₂S of cross-linker). Dh (DLS)=25±10 nm; PDI (size) 0.6.

### B.3. Functionalization of DXT-A-SCPN with 3-Mercaptopropionic Acid (DXT-A-SCPN1-F1)

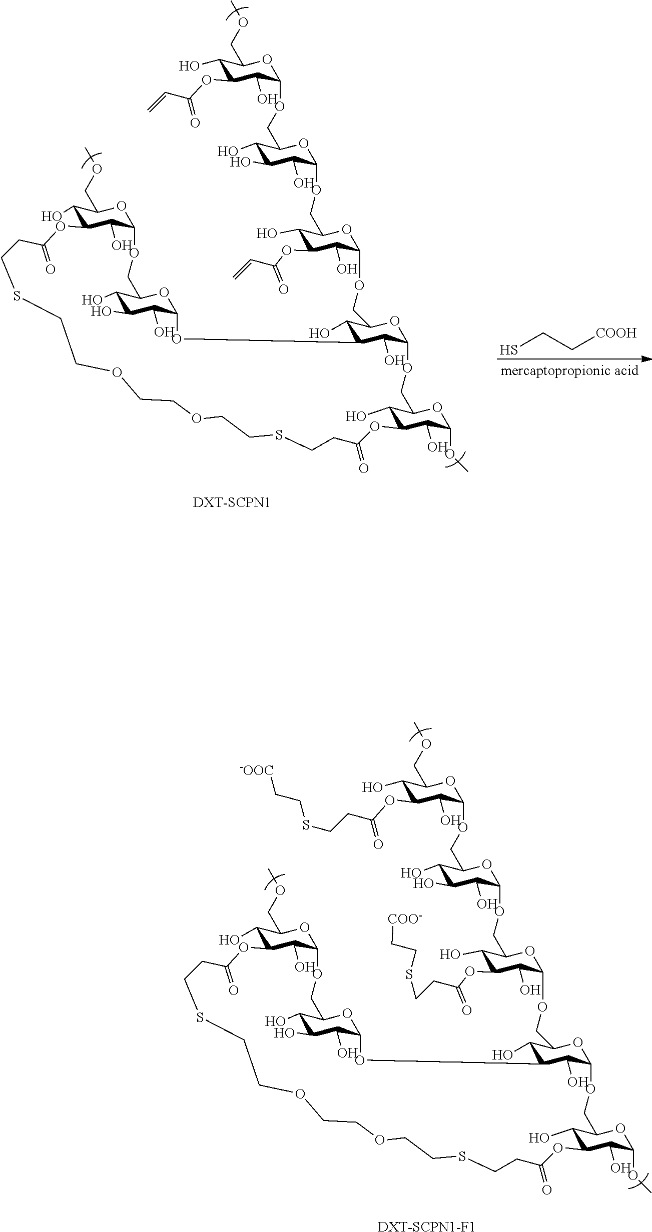

### Example 19. DXT-A-SCPN1-F1 (from DXT-A-SCPN1 of Example 18)

The synthesis of the functionalized DXT-A-SCPN1-F1 was achieved by adding slowly 0.5 mL of a freshly prepared aqueous solution of 3-mercaptopropionic acid (37 μL, 463 μL H₂O, pH=8) the reaction flask in the DXT-A-SCPN1 obtained in Example 18. The reaction was stirred for 16 h and the excess acid was removed by dialysis against distilled water (MWCO 3.5 kDa) until reaching deionized water conductivity values <1 μS·cm⁻¹ (5 days, refreshing with 4 L of deionized water twice per day). The resulting aqueous solution was freeze-dried to obtain functionalized nanoparticles DXT-A-SCPN1-F1 as a white solid. Yield >90%. ¹H NMR (500 MHz, D₂O) δ ppm: 5.35-4.90 (4.1H, including H-1 and H-2/3 A-substituted Glc protons), 4.05-3.46 (20.5H, m, rest of Glc and 2×CH₂O of crosslinker), 2.81-2.70 (7.3H, m, CH(CH₃)CH₂S, CH₂S of cross-linker and quenching linker), 2.39-2.43 (2.1H, m, CH₂COOH). GPO-SEC characterization for the DXT-A-SCPN1-F1 was carried out and the DXT-A-SCPN1-F1 was compared with DXT-A-F1 (obtained by reacting DXT-A of Example 17 with mercaptopropionic acid following the process as defined in Example 19) to confirm the increase of the retention time due to the reduction of the hydrodynamic radius as evidence of the polymer compaction into SCPN: DXT-A-SCPN1-F1, Mw (GPC)=77 KDa, Mw/Mn=1.3. Dh (DLS)=21±6 nm; PDI (size) 0.3.

### Example 20. In Vivo Pharmacokinetic Profile of Tob-DXT-SCPN1-Ionic (Example 9) in the Lungs of Sprague Dawley Rats

Samples:
Tobramycin (control)
Tob-DXT-SCPN1-ionic of the invention (Example 9)

Method

Fifty-four male Sprague Dawley rats weighing ca 289-353 g received a lung dose of either 10 mg/kg of free tobramycin or 10 mg/kg of tobramycin formulated as Tob-DXT-SCPN1-ionic of the present invention. The administration technique was oropharyngeal deposition. Following administration, terminal lung samples were collected at the following time-points: 0.083, 0.25, 0.5, 1, 2, 4, 8, 12, 24 h post-dose. The tobramycin concentration in lung homogenates was determined by LC-MS.

Results

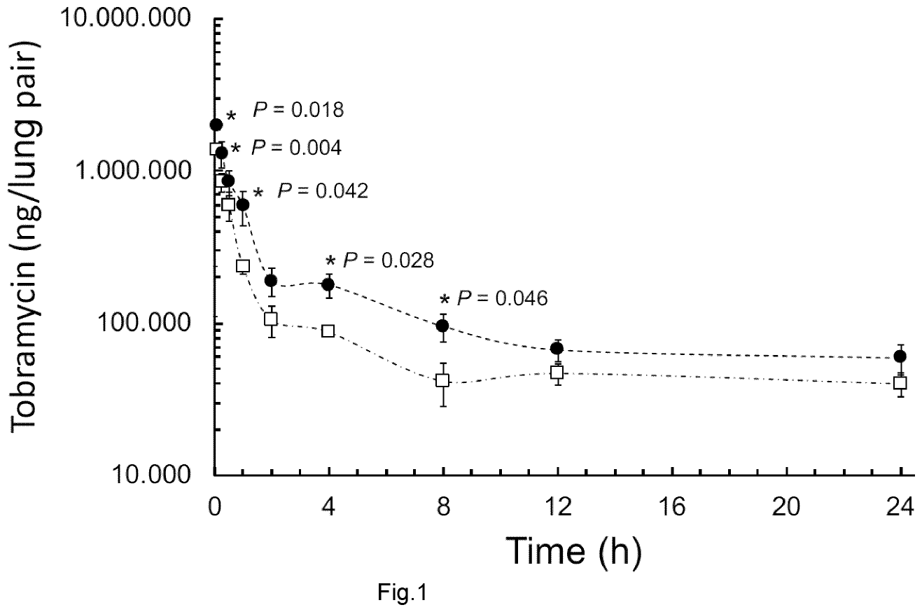

FIG. 1 shows the pharmacokinetic profile of free tobramycin (white squares) and Tob-DXT-SCPN1-ionic of the present invention (black circles). The mean tobramycin levels per lung pair were higher in those animals receiving Tob-DXT-SCPN1-ionic of the invention. Moreover, significant differences were found between animals treated either with free tobramycin or Tob-DXT-SCPN1-ionic of the invention at 0.083, 0.25, 1, 4, and 8 hours (*P<0.05. one-way ANOVA, n=3 animals per time-point and group). The Area under the curve (AUC) (obtained by trapezoidal method) was higher in the case of Tob-DXT-SCPN1-ionic of the invention (1,660,000 ng·h/mL) compared to free tobramycin (1,110,000 ng·h/mL). A higher AUC indicates a higher lung exposure of the active pharmaceutical ingredient, in this case, tobramycin.

For the purpose of the invention, the AUC was measured by the trapezoidal method using the following formula:

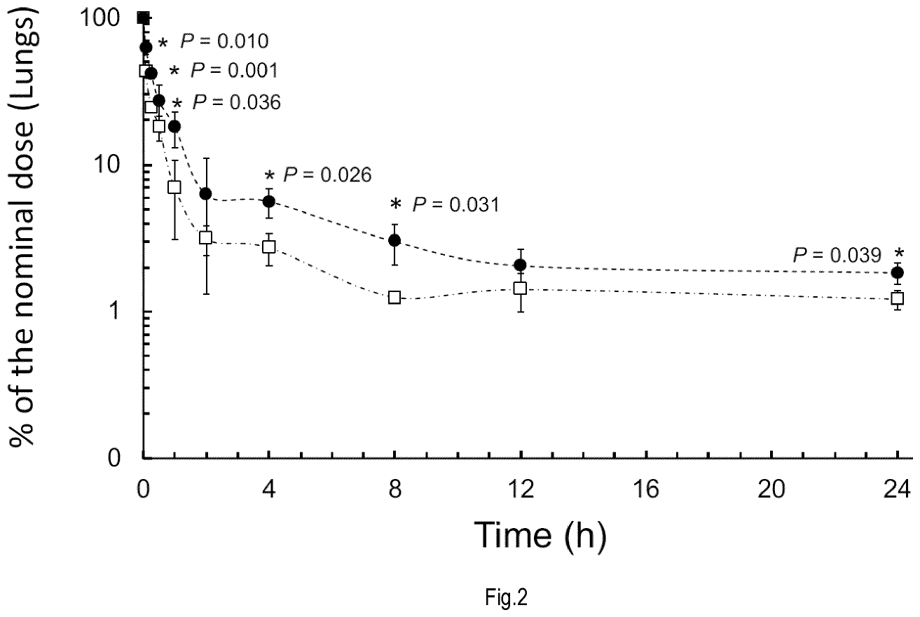

AUC0-t=(C1+C2/2)*(t2−t1)

Wherein: C1 is the tobramycin concertation at point 1, C2 is the tobramycin concentration at point 2, t1 is time of C1 and t2 is time of C2. Further, FIG. 2 shows the mean percentage of tobramycin that remains in the lungs of animals either treated with free tobramycin (white squares) or Tob-DXT-SCPN1-ionic of the present invention (black circles) as a function of the nominal dose. The “nominal dose” is equivalent to the “administered dose” and describes the individual amount of tobramycin administered to each animal (10 mg/kg). The mean percentage of the nominal dose remaining in the lungs of animals treated with Tob-DXT-SCPN1-ionic of the invention was higher in all time-points compared to the mean percentage of the animals treated with free tobramycin. Significant differences between animals treated with Tob-DXT-SCPN1-ionic of the invention and free tobramycin were detected at 0.083, 0.25, 1, 4, 8, and 24 hours after administration of the nominal dose (*P<0.05. one-way ANOVA, n=3 animals per time-point and group).

Conclusion

Water dispersible single-chain dextran methacrylate or acrylate based nanoconjugates of the present invention (particularly formulated as Tob-DXT-SCPN1-ionic of example 9 of the present invention) lead to a tobramycin release profile that increases the antibiotic lung concentration over time compared to the free tobramycin.

### Example 21. Antibiotic Susceptibility Test of the Tob-DXT-SCPN of the Present Invention with Clinical Isolates of P. aeruginosa

Samples:
Tobramycin (control)
Tob-DXT-SCPN1-ionic of the invention (Example 9)

Method:

MIC of the free hydrophilic tobramycin and Tob-DXT-SCPN1-ionic of the present invention was performed in a panel of P. aeruginosa strains that included investigational strains and strains obtained from clinical isolates. The clinical isolates were obtained from the Biobank of the Hospital Donostia/Biodonostia (San Sebastian, Spain) with permission. MIC is obtained by visual inspection and considered the minimum concentration at which no bacterial growth can be detected (i. e. the buffer appears transparent) when bacteria was incubated at gradient concentration of test compound.

Results:

The results are summarized in the table below:

| | Test Article MIC (μg/mL) | | |
|---|---|---|---|
| | Free | Tob-DXT- | |
| | Tobramycin | CPN1-ionic | |
| Strain | (control) | of the invention | Features |
| P. aeruginosa | 0.5^{a} | 0.5 | Reference strain |
| ATCC 27853 | | | |
| PAO1 | 0.5 | 0.5 | Investigational |
| DSMZ 22644 | | | strain |
| PA14 | 0.5 | 0.5 | Investigational |
| DSMZ 19882 | | | strain |
| PACC#5 | 0.5 | 0.5 | Clinical isolate, |
| | | | mucoid phenotype |
| PACC#6 | ≥4 | ≥4 | Clinical isolate, low |
| | | | susceptibility for |
| | | | tobramycin |
| PACC#7 | 0.5 | 0.5 | Clinical isolate, |
| | | | mucoid phenotype |
| PACC#8 | 0.5 | 0.5 | Clinical isolate |
| PACC#9 | 0.5 | 0.5 | Clinical isolate, |
| | | | mucois phenotype |
| PACC#10 | 1 | 1 | Clinical isolate, |
| | | | mucoid phenotype |

^{a}Within CLSI M100 S27 QC range (0.25-1 μg/mL)
PACC: Pseudomonas aeruginosa Clinical Case

Conclusion:

The above-disclosed results confirm that the antibiotic efficacy of the Tob-DXT-SCPN1-ionic of the present invention (in investigational P. aeruginosa strains and in strains isolated from hospitalized patientsis) is comparable to the free Tobramycin. It means that the water-dispersible single-chain dextran methacrylate or acrylate based nanoconjugates of the present invention are therapeutically efficient for being use in therapy.

1. A water-dispersible single-chain dextran methacrylate or acrylate based nanoconjugate, which comprises:
a water-dispersible single-chain dextran methacrylate or acrylate based nanoparticle and
one or more hydrophilic active ingredients,
wherein the nanoconjugate comprises units of formula (I) 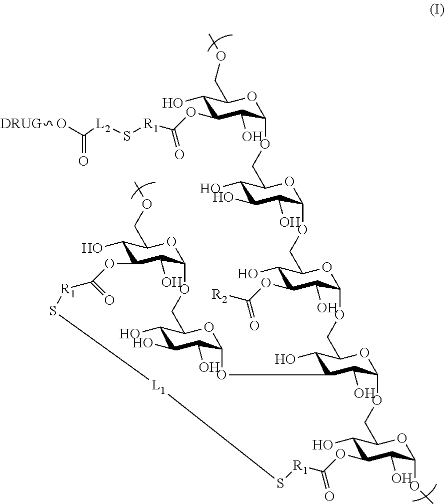
wherein:
R₁ is selected from the group consisting of —CH₂—CH₂— and —CH₂—CH(CH₃)—;
R₂ is selected from the group consisting of —CH(═CH₂), —C(═CH₂)(CH₃), —CH₂—CH₂—S—(CH₂)ᵥ—COOH, —CH(CH₃)—CH₂—S—(CH₂)ᵥ—COOH, —CH₂—CH₂—S—[(CH₂)_{w}—O]ₓ—(CH₂)_{w}—OH, and —CH(CH₃)—CH₂—S—[(CH₂)_{w}—O]ₓ—(CH₂)_{w}—OH;
L₁ is a biradical selected from the group consisting of —[(CH₂)ᵣ—O]_{q}—(CH₂)ᵣ— and —(CH₂)ₛ—;
L₂ is a biradical selected from the group consisting of —(CH₂)ᵥ— and —(C₅-C₆)arylene-NH—CO—(CH₂)ₚ—;
DRUG is the hydrophilic active ingredient;
p and v are integers from 1 to 6;
w is an integer from 2 to 4;
x is an integer from 0 to 4;
q is an integer from 2 to 3;
r is an integer from 2 to 4;
s is an integer from 2 to 5; [symbol]
represents a non-covalent bond between the negative charge of the oxygen atom of the group —O—CO— of the nanoparticle and the positive charge of a nitrogen atom of the drug; or alternatively, a hydrolysable covalent bond between the oxygen atom of the group —O—CO— of the nanoparticle and a carbon atom of the drug; and
wherein the nanoconjugate has:
a particle size from 3 nm to 50 nm measured by Dynamic Light Scattering;
a zeta potential from −25 mV to +20 mV;
a percentage of intra-molecular crosslinking from 3% to 45% molar % of the total amount of monomer units present in the dextran methacrylate or acrylate chain; and
a drug loading from 20% to 40% by weight of the drug in relation to the total weight of the nanoconjugate.

2. The water-dispersible single-chain dextran methacrylate or acrylate based nanoconjugate according to claim 1, wherein the hydrophilic active ingredient is a hydrophilic antibiotic.

3. The water-dispersible single-chain dextran methacrylate or acrylate based nanoconjugate according to claim 1, wherein: [symbol]
represents a non-covalent bond;
L₁ is —[(CH₂)ᵣ—O]_{q}—(CH₂)ᵣ—;
L₂ is —(CH₂); and
(i) R₁ is —CH₂—CH₂—; and
R₂ is —CH₂—CH₂—S—(CH₂)ᵥ—COOH;
or alternatively,
(ii) R₁ is —CH₂—CH(CH₃)—; and
R₂ is —CH(CH₃)—CH₂—S—(CH₂)ᵥ—COOH.

4. The water-dispersible single-chain dextran methacrylate or acrylate based nanoconjugate according to claim 3, wherein:
L₁ is —(CH₂)₂—O—(CH₂)₂—O—(CH₂)₂—; and
L₂ is —(CH₂)₂—.

5. The water-dispersible single-chain dextran methacrylate or acrylate based nanoconjugate according to claim 1, wherein: [symbol]
represents a hydrolysable covalent bond;
L₁ is —[(CH₂)ᵣ—O]_{q}—(CH₂)ᵣ—;
L₂ is —(C₅-C₆)arylene-NH—CO—(CH₂)ₚ—; and
(i) R₁ is —CH₂—CH₂—; and
R₂ is —CH(═CH₂) or —CH₂—CH₂—S—[(CH₂)_{w}—O]ₓ—(CH₂)_{w} OH;
or alternatively;
(ii) R₁ is —CH₂—CH(CH₃)—; and
R₂ is —CH(═CH₂)(CH₃) or —CH(CH₃)—CH₂—S—[(CH₂)_{w}—O]ₓ—(CH₂)_{w} OH.

6. The water-dispersible single-chain dextran methacrylate or acrylate based nanoconjugate according to claim 5, wherein:
L₁ is —(CH₂)₂—O—(CH₂)₂—O—(CH₂)₂—; and
L₂ is

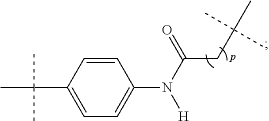

7. The water-dispersible single-chain dextran methacrylate or acrylate based nanoconjugate according to claim 5, wherein:
L₁ is —(CH₂)₂—O—(CH₂)₂—O—(CH₂)₂—; and
L₂ is

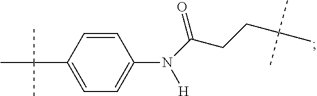

8. The water-dispersible single-chain dextran methacrylate or acrylate based nanoconjugate according to claim 1, wherein the DRUG is a hydrophilic antibiotic selected from the group consisting of aminoglycoside antibiotics, polymyxins, glycopeptides, and antimicrobial peptides.

9. A process for the preparation of the water-dispersible single-chain dextran methacrylate or acrylate based nanoconjugate according to claim 1, wherein when [symbol] represents a hydrolysable covalent bond, then the process comprises:
a) reacting a single-chain dextran methacrylate or acrylate based nanoparticle having a particle size from 3 to 50 nm, a polydispersion of the nanoparticle equal to the polydispersion of dextran ±20%; and an intra-molecular crosslinking from 3 to 45 molar % of the total amount of monomer units present in the dextran methacrylate or acrylate chain with a compound of formula
DRUG-O—CO-L₂-SH
at a pH from 7.5 to 10 and at a temperature from 20° C. to 25° C. to obtain a water-dispersible single-chain dextran methacrylate or acrylate based nanoconjugate as defined in claim 1 wherein R₂ is selected from the group consisting of —CH(═CH₂) and —C(═CH₂)(CH₃); and
b) optionally reacting the water-dispersible single-chain dextran methacrylate or acrylate based nanoconjugate obtained in claim a) with a compound of formula SH—[(CH₂)_{w}—O]ₓ—(CH₂)_{w} OH under such reaction conditions to obtain a water-dispersible single-chain dextran methacrylate or acrylate based nanoconjugate as defined in claim 1 wherein R₂ is selected from the group consisting of —CH₂—CH₂—S—[(CH₂)_{w}—O]ₓ—(CH₂)_{w} OH, and —CH(CH₃)—CH₂—S—[(CH₂)_{w}—O]ₓ—(CH₂)_{w}—OH;
or alternatively,
wherein when [symbol] represents a non-covalent bond, then the process comprises:
c) reacting a single-chain dextran methacrylate or acrylate based nanoparticle having a particle size from 3 to 50 nm, a polydispersion of the nanoparticle equal to the polydispersity of dextran ±20%; and an intra-molecular crosslinking from 3 to 45 molar % of the total amount of monomer units present in the dextran methacrylate or acrylate chain with a compound of formula
HS-L₂-COOH
and
d) contacting the single-chain dextran methacrylate or acrylate based nanoparticle obtained in step c) with the DRUG and adjusting the pH to 7 by the addition of an acid to obtain the water-dispersible single-chain dextran methacrylate or acrylate based nanoconjugate as defined in claim 1, wherein R₂ is selected from the group consisting of —CH₂—CH₂—S—(CH₂)ᵥ—COOH and —CH(CH₃)—CH₂—S—(CH₂)ᵥ—COOH;
and
optionally, the process further comprises an additional step of purifying the water-dispersible single-chain dextran methacrylate or acrylate based methacrylate or acrylate nanoconjugate, and
optionally, the process further comprises an additional step of drying the water-dispersible single-chain dextran methacrylate or acrylate based nanoconjugate.

10. The process for the preparation of the water-dispersible single-chain dextran methacrylate or acrylate based nanoconjugate according to claim 9, wherein the process further comprises preparing the single-chain dextran methacrylate or acrylate based nanoparticle having a particle size from 3 to 50 nm, a polydispersion of the nanoparticle equal to the polydispersity of dextran±20%; and an intra-molecular crosslinking from 3 to 45 molar % of the total amount of monomer units present in the dextran methacrylate or acrylate chain by a process which comprises cross-linking a dextran methacrylate or acrylate having a solubility equal to or higher than 100 mg per litre of water, and an amount of methacrylate or acrylate groups from 5 to 60 molar % of the total amount of monomer units present in the dextran chain; with a homobifunctional crosslinking agent having crosslinkable groups; by
(e1) adding an aqueous solution of dextran methacrylate or acrylate having an amount of methacrylate or acrylate groups from 5 to 60 molar % of the total amount of monomer units present in the dextran chain to an aqueous solution of the homobifunctional crosslinking agent at an addition rate that allows having during the addition of dextran methacrylate or acrylate a concentration of the crosslinkable groups of the dextran methacrylate or acrylate in the solution of the homobifunctional crosslinking agent from 5·10⁻³ to 10⁻⁷ molar; or alternatively,
(e2) adding an aqueous solution of the homobifunctional crosslinking agent to an aqueous solution of the dextran methacrylate or acrylate having an amount of methacrylate or acrylate groups from 5 to 60 molar % of the total amount of monomer units present in the dextran chain at an addition rate that allows having during the addition of the homobifunctional crosslinking agent a concentration of the homobifunctional crosslinking agent in the solution of dextran methacrylate or acrylate from 5·10⁻³ to 10⁻⁷ molar; or alternatively,
(e3) mixing a solution of the homobifunctional crosslinking agent and an aqueous solution of the dextran methacrylate or acrylate having an amount of methacrylate or acrylate groups from 5 to 60 molar % of the total amount of monomer units present in the dextran chain having a concentration of the homobifunctional crosslinking agent in the mixture from 10⁻⁷ to 5·10⁻³ molar;
at a temperature from 20 to 25° C. in the absence of a catalyst;
and
optionally the process further comprises an additional step of purifying the water-dispersible single-chain dextran methacrylate or acrylate based nanoparticle,
and
optionally the process further comprises an additional step of drying the water-dispersible single-chain dextran methacrylate or acrylate based nanoparticle.

11. A pharmaceutical composition comprising the water-dispersible dextran methacrylate or acrylate based nanoconjugate as defined in claim 1, together with one or more pharmaceutically acceptable excipients or carriers.

12. The pharmaceutical composition according to claim 11, wherein the pharmaceutical composition is an inhalation composition.

13. A method of treatment comprising using the water-dispersible dextran methacrylate or acrylate based nanoconjugate according to claim 1.

14. A method of treatment of bacterial infection in a patient, the method comprising using the water-dispersible dextran methacrylate or acrylate based nanoconjugate according to claim 1, wherein the hydrophilic active ingredient is a hydrophilic antibiotic.

15. The method of treatment according to claim 14, wherein the bacterial infection is a lower respiratory tract bacterial infection, and the patient is selected from patients with lung infections who suffer from chronic obstructive pulmonary diseases, cystic fibrosis, bronchoectasia, and hospital induced pneumonia.

16. The water-dispersible single-chain dextran methacrylate or acrylate based nanoconjugate according to claim 2, wherein: [symbol]
represents a non-covalent bond;
L₁ is —[(CH₂)ᵣ—O]_{q}—(CH₂)ᵣ—;
L₂ is —(CH₂)ᵥ;
(i) R₁ is —CH₂—CH₂—; and
R₂ is —CH₂—CH₂—S—(CH₂)ᵥ—COOH;
or alternatively,
(ii) R₁ is —CH₂—CH(CH₃)—; and
R₂ is —CH(CH₃)—CH₂—S—(CH₂)ᵥ—COOH.

17. The water-dispersible single-chain dextran methacrylate or acrylate based nanoconjugate according to claim 16, wherein:
L₁ is —(CH₂)₂—O—(CH₂)₂—O—(CH₂)₂—; and
L₂ is —(CH₂)₂—.

18. The water-dispersible single-chain dextran methacrylate or acrylate based nanoconjugate according to claim 2, wherein: [symbol]
represents a hydrolysable covalent bond;
L₁ is —[(CH₂)ᵣ—O]_{q}—(CH₂)ᵣ—;
L₂ is —(C₅-C₆)arylene-NH—CO—(CH₂)ₚ—;
(i) R₁ is —CH₂—CH₂—; and
R₂ is —CH(═CH₂) or —CH₂—CH₂—S—[(CH₂)_{w}—O]ₓ—(CH₂)_{w} OH;
or alternatively,
(ii) R₁ is —CH₂—CH(CH₃)—; and
R₂ is —CH(═CH₂)(CH₃) or —CH(CH₃)—CH₂—S—[(CH₂)_{w}—O]ₓ—(CH₂)_{w} OH;
and
p is an integer from 1 to 6;
q is an integer from 2 to 3;
r is an integer from 2 to 4;
x is an integer from 0 to 4; and
w is an integer from 2 to 4.

19. The water-dispersible single-chain dextran methacrylate or acrylate based nanoconjugate according to claim 18, wherein:
L₁ is —(CH₂)₂—O—(CH₂)₂—O—(CH₂)₂—; and
L₂ is

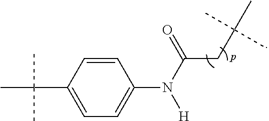

20. The water-dispersible single-chain dextran methacrylate or acrylate based nanoconjugate according to claim 6 wherein: [symbol]
represents a hydrolysable covalent bond;
L₁ is —(CH₂)₂—O—(CH₂)₂—O—(CH₂)₂—;
L₂ is 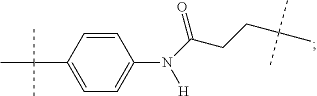
(i) R₁ is —CH₂—CH₂—; and
R₂ is —CH(═CH₂) or —CH₂—CH₂—S—[(CH₂)₂—O]₂—(CH₂)₂—OH,
or alternatively,
(ii) R₁ is —CH(CH₃)—CH₂—; and
R₂ is —C(═CH₂)(CH₃) or —CH(CH₃)—CH₂—S—[(CH₂)₂—O]₂—(CH₂)₂—OH.
